(19) United States
(12) Patent Application Publication
(10) Pub. No.: US 2010/0050301 A1
(43) Pub. Date: Feb. 25, 2010

(54) SYSTEM FOR CAPTURING AND MODIFYING LARGE PIECES OF GENOMIC DNA AND CONSTRUCTING VASCULAR PLANTS WITH SYNTHETIC CHLOROPLAST GENOMES

(75) Inventors: MICHAEL MENDEZ, SAN DIEGO, CA (US); BRYAN O'NEILL, SAN DIEGO, CA (US); KARI MIKKELSON, SAN DIEGO, CA (US)

Correspondence Address: Sapphire Energy, 3115 Merryfield Row, San Diego, CA, 92121, US

(73) Assignee: SAPPHIRE ENERGY, INC., SAN DIEGO, CA (US)

(21) Appl. No.: 12/560,341

(22) Filed: Sep. 15, 2009

Related U.S. Application Data
(63) Continuation-in-part of application No. 12/384,893, filed on Apr. 8, 2009.
Application No. 12/384,893 is a continuation-in-part of application No. 12/287,230, filed on Oct. 6, 2008.
(60) Provisional application No. 60/978,024, filed on Oct. 5, 2007.

(51) Int. Cl. A01H 5/00; C12N 15/74; C12N 15/64; C12N 1/21; C12N 1/19; C07H 21/04

(52) U.S. Cl. 800/298; 435/320.1; 435/91.42; 435/252.3; 435/254.2; 536/23.6

(57) ABSTRACT

Vectors capable of stable replication in yeast and bacteria and comprising all essential genes of vascular plant plastids are provided as well as the use of such vectors to construct an recombinant plastid genome and host cells transformed with said vectors and recombinant plastid genomes.

### CROSS REFERENCE TO RELATED APPLICATIONS

The present application is a continuation in part of copending U.S. patent application Ser. No. 12/384,893 filed Apr. 8, 2009, which is a continuation in part of copending U.S. patent application Ser. No. 12/287,230 filed Oct. 6, 2008, which claims the benefit of U.S. Provisional Patent Application 60/978,024 filed Oct. 5, 2007, now abandoned; each of which is incorporated by reference in its entirety for all purposes.

### BACKGROUND

For the functional analysis of many genes, investigators need to isolate and manipulate large DNA fragments. The advent of genomics and the study of genomic regions of DNA have generated a need for vectors capable of carrying large DNA regions.

In general, two types of yeast vector systems are presently available. The first type of vector is one capable of transferring small insert DNA between yeast and bacteria. A second type of vector is a fragmenting vector which creates interstitial or terminal deletions in yeast artificial chromosomes (YACs). The small insert shuttle vectors are able to recombine with and recover homologous sequences. They are centromere-based and replicate stably and autonomously in yeast, but also contain a high-copy origin of replication for maintenance as bacterial plasmids. However, these vectors are limited by their small insert capacity. The second type of vector (also known as fragmenting vectors) has recombinogenic sequences, but is unable to transfer the recovered insert DNA to bacteria for large preparations of DNA.

Researchers use fragmentation techniques to narrow down the region of interest in YACs. Isolating sufficient quantities of YAC DNA from agarose gels for microinjection or electroporation, however, remains cumbersome. Purification remains a problem when the YAC comigrates with an endogenous chromosome. In addition, YACs may be chimeric or contain additional DNA regions that are not required for the particular functional study.

Types of vectors available for cloning large fragments in bacteria are cosmids, P1s and bacterial artificial chromosomes (BACs). These vectors are limited to bacteria and cannot be shuttled to yeast for modification by homologous recombination. Bacterial vectors are also limited in their use for transforming plants. For example, although chloroplasts are thought to originate from the endosymbiosis of photosynthetic bacteria into eukaryotic hosts, translation in chloroplasts is more complex. Adding to the complexity of genetically engineering plants is the presence of multiple chloroplasts with multiple copies of the chloroplast genome. Thus, there exists a need for developing a method to express proteins from large fragments of DNA in the chloroplasts of plants.

### SUMMARY

Disclosed herein are compositions and methods of isolating, characterizing, and/or modifying large DNA, including entire genomes of chloroplasts. The compositions include shuttle vectors into which target DNA may be inserted. The methods include modifying or manipulating target DNA by removing, adding or rearranging portions and introducing the modified DNA into a host, in particular a vascular plant.

One aspect provides an isolated vector comprising a yeast element, a bacterial origin of replication, and at least 90% of the plastid genome of a vascular plant. In some vectors, the yeast element is a yeast centromere, a yeast autonomous replicating sequence, yeast auxotrophic marker, or a combination thereof. The plastid genome may be from, for example, soybean (G. max), arabidopsis (A. thaliana), corn (Z. mays), rice (O. sativa) or wheat (T. aestivum). In some embodiments, the plastid genomic DNA is modified, for example by insertion of a heterologous or homologous polynucleotide, deletion of one or more nucleic acid bases, mutation of one or more nucleic acid bases, rearrangement of one or more polynucleotides, or a combination thereof. In some instances, the modification is synthetic. Vectors of the present disclosure, when transformed into a plant host cell, may result in production of a product not naturally produced by the host plant cell. The vectors disclosed herein may further comprise one or more selection markers, for example, a yeast marker, a yeast antibiotic resistance marker, a yeast auxotrophic marker, a bacterial marker, a bacterial antibiotic resistance marker, a bacterial auxotrophic marker or any combination thereof. Vectors may also contain chloroplast genomic DNA which comprises 1) 1-200 genes; 2) all essential chloroplast genes; 3) at least 90% of a chloroplast genome; 4) at least 135 kb; or 5) at least 150 kb.

Also described is a plant host cell comprising the vectors described herein. Exemplary host cells may be monocots or dicots and include, but are not limited to, soybeans, tomatoes, potatoes, wheat, rice, corn, barley, rye, and cotton.

Another aspect provides method for producing a vector where the method involves inserting targeting DNA into a vector—where the vector comprises a yeast centromere, a yeast autonomous replicating sequence, and a bacterial origin of replication, transforming an organism, for example yeast, with the vector and capturing a portion of a chloroplast genome, thus producing a vector with a portion of or a complete chloroplast genome. In some instances, the targeting DNA is chloroplast genomic DNA. This method may be used to capture a portion of a genome which is at least 135 kb in length, at least 150 kb or that comprises at least 90% of a chloroplast genome. In some instances, the capturing step occurs by recombination. The captured portion of a chloroplast genome may be co-transformed into an organism with a vector, thus the recombination step may occur in vivo. Vascular plants used to practice methods disclosed herein may be monocots or dicots and include the major agricultural crops. In some embodiments, an additional step of modifying a portion of a chloroplast genome is utilized. A modification may be achieved through homologous recombination. Such recombination may occur in an organism, for example yeast. In embodiments with a modification step, the step may comprise addition of a polynucleotide, deletion of one or more nucleic acid bases, mutation of one or more nucleic acid bases, rearrangement of a polynucleotide, or any combination thereof.

Further disclosed herein is an isolated vector comprising essential chloroplast genes, a selectable marker and a manipulation in one or more nucleic acids in the vector. In some instances, essential chloroplast genes are cloned from a vascular plant such as soybean (G. max), arabidopsis (A. thaliana), corn (Z. mays), rice (O. sativa) or wheat (T. aestivum). Essential chloroplast genes for use in the vectors described herein may be synthetic. The vectors described herein may further comprise an expression cassette, which may further comprise a region for integration into target DNA, for example plastid DNA. The vectors described herein may also contain one or more selection markers, for example, an auxotrophic marker, an antibiotic resistance marker, a chloroplast marker, or any combination thereof. In some instances, the essential chloroplast genes are those required for chloroplast function, photosynthesis, carbon fixation, production of one or more products, or any combination thereof. Essential chloroplast genes may comprise up to 200 genes and/or consist of up to 400 kb. In some of the vectors described herein, a manipulation in one or more nucleic acids is an addition, deletion, mutation, or rearrangement. In some instances, expression of the vector in a host cell produces a product not naturally produced by said host cell. In other instances, expression of a vector of the present invention results in an increase production of a product naturally produced by said host cell.

One aspect provides an isolated chloroplast comprising a vector described herein. In another aspect, a host cell comprising a vector described herein is provided. Host cells useful in the present include those obtained from any vascular plant. Examples of plants useful for the present disclosure include soybeans, tomatoes, potatoes, wheat, rice, corn, barley, rye, and cotton.

In another aspect, a method for transforming a cell or organism is provided where the method comprises inserting into said cell a vector comprising all essential chloroplast genes or 90% of a chloroplast genome. Optionally, the vector can comprise one or more genes not naturally occurring in said cell or organism. In some embodiments, the method further comprises the step of eliminating substantially all chloroplast genomes in said cell or organism or disabling the photosynthetic capability of the cell. A cell or organism useful for this method may be photosynthetic, non-photosynthetic and/or eukaryotic. In some instances, the vector for use in this method may also comprise an expression cassette and the expression cassette may be capable of integrating into non-nuclear DNA. In one embodiment the one or more genes not naturally occurring in the cell or organism is a gene in the isoprenoid pathway, MVA pathway, or MEP pathway. In another embodiment, the essential chloroplast genes are those that are required for chloroplast function, photosynthesis, carbon fixation, production of one or more hydrocarbons, or a combination thereof. In still another embodiment, the genes not naturally occurring in the cell or organism confer herbicide, insect and/or disease resistance. In another embodiment, the one or more genes not naturally occurring in the cell or organism produce a therapeutic protein. In another embodiment, the one or more genes not naturally occurring in the cell or organism increase production of a lipid, fatty acid or phytosterol.

Further provided herein is a method for modifying an organism comprising the steps of transforming the organism, and in particular a vascular plant, with a vector disclosed herein. In some instances, a vector useful for this method further comprises a sequence for production and/or secretion of a compound from said organism. In other instances, the vector comprises all essential chloroplast genes. In other instances, the vector comprises at least 90% of a chloroplast genome. In still other instances, the essential chloroplast genes are rearranged or mutated. An organism useful for some embodiments comprises essentially no chloroplast genome or a chloroplast genome incapable of photosynthesis prior to transformation.

Yet another method provided herein is a method for making a product from an organism comprising the step of transforming said organism with a vector described herein and further comprising one or more of the following: (i) a gene not naturally occurring in said organism; (ii) a deletion in a gene naturally occurring in said organism; (iii) a rearrangement of genes naturally occurring in said organism; and (iv) a mutation in a gene naturally occurring in said organism. In some instances, the organism is naturally photosynthetic. In other instances, the additional genes encode enzymes in the isoprenoid pathway, MVA pathway, or MEP pathway. In other instances, the additional genes allow for the production of therapeutic products, such as therapeutic proteins and phytosterols. In still another embodiment, the present disclosure provides a method for transforming a cell or organism comprising inserting into said cell or organism a chloroplast and a vector comprising all essential chloroplast genes.

The present disclosure also provides a method of producing an artificial chloroplast genome comprising the steps of: (a) providing a vector comprising one or more essential chloroplast genes; (b) adding to said vector a DNA fragment; (c) transforming a cell or organism with the vector produced by step (b); and (d) determining whether chloroplast function exists with said added DNA fragment. In some instances, the added DNA fragments comprise one or more coding regions for an enzyme in the isoprenoid, MVA or MEP pathway.

The present disclosure also provides a shuttle vector comprising at least 90% of a chloroplast genome of a vascular plant. In some instances, the genome may be modified. The shuttle vector comprises at least one selection marker, at least one yeast element, at least one bacterial origin of replication and at least one bacterial selection marker. The shuttle vector is capable of stable replication in yeast and bacterial cells. In certain embodiments, the yeast elements are a yeast centromere sequence, a yeast autonomously replicating nucleotide sequence or both. In other embodiments the bacterial origin of replication is a P1 or F′ origin of replication. Also provided herein is a vector comprising an isolated, functional chloroplast genome. A chloroplast genome useful in such a vector may be modified.

Further provided herein is a method of producing an artificial chloroplast genome comprising the steps of: (a) providing a vector comprising all essential chloroplast genes; and (b) removing, adding, mutating, or rearranging DNA from the chloroplast genome. Such a method may further comprise the steps of transforming a redacted genome into a host organism; and (d) determining chloroplast function in the host organism. In some instances, steps (b), (c), and (d) are repeated. In still other instances, the chloroplast genome is from an organism selected from the group consisting of: soybeans, tomatoes, potatoes, wheat, rice, corn, barley, oats, rye, cotton, Arabidopsis, tobacco, and legumes (e.g., peas, beans, lentils, alfalfa, etc.). For some embodiments, the method may further comprise the step of removing redundant DNA from a chloroplast genome. In other embodiments, the vector comprises all or substantially all of a chloroplast genome, for example at least 85%, at least 87%, at least 90%, at least 92% or at least 95% of a chloroplast genome. A chloroplast genome useful in the present invention may be cloned from a photosynthetic organism or may be a synthetic chloroplast genome. In some instances, the vector further comprises a gene not naturally occurring in the host organism.

Yet another method provided herein is a method of producing an artificial chloroplast genome comprising the steps of: (a) providing a vector comprising an entire chloroplast genome; (b) deleting a portion of said entire chloroplast genome; and (c) determining whether chloroplast function exists without said deleted portion. In another aspect of the present invention, a composition comprising an isolated and functional chloroplast genome is provided. In some instances, a composition comprises a modification to said chloroplast genome.

Further provided herein is an ex vivo vector comprising a nucleic acid comprising at least about 85%, at least about 87%, at least about 90%, at least about 92%, at least about 95%, or all essential genes of a chloroplast genome and a manipulation in one or more nucleic acids in the vector. In some instances, the vector comprises at least about 135 kb or at least about 150 kb of chloroplast genomic DNA. In some instances, the nucleic acid is cloned from a vascular plant, for example soybeans, tomatoes, potatoes, wheat, rice, corn, barley, oats, rye, cotton, Arabidopsis, tobacco, and legumes. In some instances, the nucleic acid is synthetic. A vector of the present disclosure may further comprise an expression cassette and an expression cassette may further comprise a region for integration into target DNA. In some instances, the target DNA is organelle DNA. A vector useful in the present disclosure may further comprise one or more selection markers, for example an auxotrophic marker, an antibiotic resistance marker, a chloroplast marker, or combinations thereof. In some embodiments, a manipulation in one or more nucleic acids in a vector may be an addition, deletion, mutation, or rearrangement. Expression of the vector may result in production of a product not naturally produced by a host cell and/or an increase production of a product naturally produced by a host cell. Examples of some products of the present invention include a terpene, terpenoid, fatty acid, a biomass degrading enzyme, a therapeutic protein and/or a phytosterol.

Further provided herein is a method of producing a vector containing a reconstructed genome, comprising: introducing two or more vectors into a host cell, wherein the vectors comprise fragments of a genome, recombining the vectors into a single vector comprising at least about 85%, at least about 87%, at least about 90%, at least about 92% or at least about 95% of a plastid genome, thereby producing a vector containing a reconstructed genome. In some instances, the host cell is eukaryotic, for example, a plant cell. The plastid may be a chloroplast, for example a chloroplast from a vascular plant such as soybeans, tomatoes, potatoes, wheat, rice, corn, barley, oats, rye, cotton, Arabidopsis, tobacco, and legumes. In some instances, the two or more vectors comprise a selectable marker. In other instances, at least one of said fragments is synthetic. In still other instances, a further step comprising modifying a portion of the genome is useful in this method. Such a modification may comprise an addition, deletion, mutation, or rearrangement. In other embodiments, the modification is the addition of an exogenous nucleic acid which results in the production or increased production of a terpene, terpenoid, fatty acid or biomass degrading enzyme.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides a general description of a hybrid vector of the present invention. FIG. 1A) Vector schematic. FIG. 1B) DNA shuttling between organisms.

FIG. 2 is a schematic showing clone 04E08.

FIG. 3 shows analysis of the G. max chloroplast genome cloned into the hybrid vector system. FIG. 3A) PCR screen data of isolated yeast transformants using primers 6113 and 6114. FIG. 3B) PCR screen data of Gm-001 in its isolated yeast clone using 12 different PCR primer pairs (lane 1, 6113 and 6114; lane 2, 6115 and 6116: lane 3, 6117 and 6118; lane 4, 6119 and 6120; lane 5, 6121 and 6122; lane 6, 6123 and 6124; lane 7, 6125 and 6126; lane 8, 6127 and 6128; lane 9, 6129 and 6130; lane 10, 6131 and 6132; lane 11, 6133 and 6134; and lane 12, 6135 and 6136). FIG. 3C) PCR screen data of Gm-001 in bacteria using 14 different PCR primer pairs (lane 1, 6113 and 6114; lane 2, 6115 and 6116: lane 3, 6117 and 6118; lane 4, 6119 and 6120; lane 5, 6121 and 6122; lane 6, 6123 and 6124; lane 7, 6125 and 6126; lane 8, 6127 and 6128; lane 9, 6129 and 6130; lane 10, 6131 and 6132; lane 11, 6133 and 6134; lane 12, 6135 and 6136, lane 13, 6105 and 6106; and lane 14, 6107 and 6108). FIG. 3D shows the stability of the isolated clone in the hybrid system.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of yeast genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al. “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This invention teaches methods and describes tools for capturing and modifying large pieces of DNA. It is especially useful for modifying genomic DNA, including the whole genome of an organism or organelle, or a part thereof. Novel prophetic uses of the invention are also described. The disclosure relates to the manipulation and delivery of large nucleic acids. The disclosure further relates to recombinational cloning vectors and systems and to methods of using the same.

Contemporary methods for genetically engineering genomes (e.g., chloroplast genomes) are time intensive (>1 month) and allow for only a limited number of manipulations at a time. If multiple modifications to a target genome are desired, the process must be iterated, further increasing the time required to generate a desired strain. Because metabolic engineering and/or synthetic biology require numerous modifications to a genome, these technologies are not amenable to rapid introduction of modifications to a genome. Thus, a new technology that allows for multiple modification of the chloroplast genome in a short amount of time will enable the application of metabolic engineering and/or synthetic biology to chloroplast genomes. The disclosure herein describes such technology.

The instant invention provides a versatile, recombinational approach to the capture, cloning, and manipulation of large nucleic acids from target cells and organelles (e.g., chloroplasts). One aspect of the present disclosure provides a recombinational cloning system. More specifically, the disclosure provides vectors, which in some embodiments, rely on homologous recombination technologies to mediate the isolation and manipulation of large nucleic acid segments. Another aspect of the present disclosure provides methods for using such recombinational cloning vectors to clone, to manipulate and to deliver large nucleic acids to target cells and/or organelles such as chloroplasts.

In one embodiment, homologous recombination is performed in vitro. In another embodiment, homologous recombination is performed in vivo. In still another embodiment, homologous recombination occurs in a yeast cell. In one embodiment, homologous recombination occurs in Saccharomyces cerevisiae or Schizosaccharomyces pombe. In yeast, the combination of efficient recombination processes and the availability of numerous selectable markers provides for rapid and complex engineering of target DNA sequences. Once all of the modifications are made to an ex vivo vector containing chloroplast genome DNA, the entire vector can be introduced into a chloroplast in a single transformation step. Thus, employing yeast technology enables the application of metabolic engineering and/or synthetic biology to chloroplast genomes. One aspect of the present disclosure provides an isolated vector comprising a yeast element, a bacterial origin of replication, and at least about 135 kb or at least about 150 kb of plastid genomic DNA from a vascular plant. In some vectors, the yeast element is a yeast centromere, a yeast autonomous replicating sequence, or a combination thereof. The DNA may be from a vascular photosynthetic plant, for example soybeans, tomatoes, potatoes, wheat, rice, corn, barley, oats, rye, cotton, Arabidopsis, tobacco, and legumes. In some embodiments, the genomic plastid DNA is modified, for example by insertion of a heterologous or homologous polynucleotide, deletion of one or more nucleic acid bases, mutation of one or more nucleic acid bases, rearrangement of one or more polynucleotides, or a combination thereof. In some instances, the modification is synthetic. Vectors of the present disclosure, when transformed into a host cell, may result in production of a product not naturally produced by the host cell. Some examples of such products include biomass-degrading enzymes, fatty acids, terpenes, terpenoids, therapeutic proteins and/or phytosterols. In some host cells, expression of the vector results in increased production of a product naturally produced by said host cell, for example, a biomass-degrading enzyme, a terpene, a terpenoid, a therapeutic protein and/or a phytosterol. The vectors of the present invention may further comprise one or more selection markers, for example, a yeast marker, a yeast antibiotic resistance marker, a bacterial marker, a bacterial antibiotic resistance marker, a plant marker, a plant antibiotic resistance marker or a combination thereof. Vectors of the present invention may also contain chloroplast genomic DNA which comprises 1) 1-200 genes; 2) all essential chloroplast genes; and/or 3) at least about 85%, 87%, 90%, 92%, or 95% of a chloroplast genome.

Also described herein is a host cell comprising the vectors described herein. Exemplary host cells may be naturally non-photosynthetic or photosynthetic and include, for example, Saccharomyces cerevisiae, Escherichia coli, soybeans, tomatoes, potatoes, wheat, rice, corn, barley, oats, rye, cotton, Arabidopsis, tobacco, and legumes.

In another aspect, a method for producing a vector is provided where the method involves inserting targeting DNA into a vector—where the vector comprises a yeast centromere, a yeast autonomous replicating sequence, and a bacterial origin of replication, transforming an organism with the vector and capturing a portion of a chloroplast genome, thus producing a vector with a portion of a chloroplast genome. In some instances, the targeting DNA is chloroplast genomic DNA. This method may be used to capture a portion of a genome which is 10-400 kb in length. In some instances, the capturing step occurs by recombination. The captured portion of a chloroplast genome may be co-transformed into an organism with a vector, thus the recombination step may occur in vivo. Organisms used to practice methods disclosed herein may be eukaryotic and photosynthetic. In some instances, the organism is a vascular photosynthetic plant, for example soybeans, tomatoes, potatoes, wheat, rice, corn, barley, oats, rye, cotton, Arabidopsis, tobacco, and legumes. Organisms used to practice methods disclosed herein may also be non-photosynthetic, for example yeast. In some instances, a non-photosynthetic organism may contain exogenous chloroplast DNA. In some embodiments, an additional step of modifying a portion of a chloroplast genome is utilized. A modification may be achieved through homologous recombination. Such recombination may occur in an organism, for example a eukaryotic and/or photosynthetic organism. In some instances, the organism is a vascular plant, for example soybeans, tomatoes, potatoes, wheat, rice, corn, barley, oats, rye, cotton, Arabidopsis, tobacco, and legumes. In other instances, the organism may be non-photosynthetic, for example a yeast. In embodiments with a modification step, the step may comprise addition of a polynucleotide, deletion of one or more nucleic acid bases, mutation of one or more nucleic acid bases, rearrangement or a polynucleotide, or combination thereof.

Further disclosed herein is an isolated vector comprising essential chloroplast genes, a selectable marker and a manipulation in one or more nucleic acids in the vector. In some instances, essential chloroplast genes are cloned from a vascular photosynthetic organism such as macroalgae, microalgae, Ch. vulgaris, C. reinhardtii, D. salina, S. quadricanda or H. pluvalis. In other instances essential chloroplast genes are cloned from a vascular plant, for example, soybeans, tomatoes, potatoes, wheat, rice, corn, barley, oats, rye, cotton, Arabidopsis, tobacco, and legumes. Essential chloroplast genes for use in the vectors described herein may be synthetic. The vectors described herein may further comprise an expression cassette, which may further comprise a region for integration into target DNA, for example organelle DNA. The vectors described herein may also contain one or more selection markers, for example, an auxotrophic marker, an antibiotic resistance marker, a chloroplast marker, or combinations thereof. In some instances, the essential chloroplast genes are those required for chloroplast function, photosynthesis, carbon fixation, production of one or more hydrocarbons, or a combination thereof. In other instances essential chloroplast genes are those necessary to render an organism photoautotrophic. Essential chloroplast genes may comprise up to 200 genes and/or consist of up to 400 kb. In some of the vectors described herein a manipulation in one or more nucleic acids is an addition, deletion, mutation, or rearrangement. In some instances, expression of the vector in a host cell produces a product not naturally produced by said host cell. In other instances, expression of a vector of the present invention results in an increase production of a product naturally produced by said host cell. Examples of such products are biomass degrading enzymes, fatty acids, terpenes, terpenoids, therapeutic proteins and/or phytosterols.

As described herein, one aspect provides a chloroplast comprising a vector of the present disclosure. In one aspect the chloroplast is an isolated chloroplast. In another aspect, a host cell comprising a vector of the present disclosure is provided. Host cells useful in the present disclosure may be naturally non-photosynthetic or naturally photosynthetic. Examples of organisms useful for the present invention include Saccharomyces cerevisiae, Escherichia coli, soybeans, tomatoes, potatoes, wheat, rice, corn, barley, oats, rye, cotton, Arabidopsis, tobacco, and legumes.

In another aspect a method for transforming a plant or cell therefrom is provided where the method comprises inserting into said plant or cell therefrom with a vector comprising all essential chloroplast genes and optionally one or more genes not naturally occurring in said cell or organism. In some embodiments, the method further comprises the step of eliminating substantially all chloroplast genomes or disabling the photosynthetic capacity of said cell or organism prior to transformation. A cell or organism useful for this method may be photosynthetic, non-photosynthetic and/or eukaryotic. In some instances, the vector for use in this method may also comprise an expression cassette and the expression cassette may be capable of integrating into non-nuclear DNA. In one embodiment the one or more genes not naturally occurring in the cell or organism is a gene in the isoprenoid pathway, MVA pathway, or MEP pathway. In another embodiment, the essential chloroplast genes are those that are required for chloroplast function, photosynthesis, carbon fixation, production of one or more hydrocarbons, or a combination thereof.

Further provided herein is a method for modifying an organism comprising the steps of transforming the organism, for example a vascular plant, with a vector comprising one or more polynucleotides sufficient to perform chloroplast function. In some instances, a vector useful for this method further comprises a sequence for production or secretion of a compound from said organism. In other instances, the vector comprises all essential chloroplast genes. In still other instances, the essential chloroplast genes are rearranged or mutated. An organism useful for some embodiments comprises essentially no chloroplast genome prior to transformation.

Yet another method provided herein is a method for making a product from an organism comprising the step of transforming said organism with a vector comprising at least 135 kb of genomic plastid DNA and one or more of the following: (i) a gene not naturally occurring in said organism; (ii) a deletion in a gene naturally occurring in said organism; (iii) a rearrangement of genes naturally occurring in said organism; and (iv) a mutation in a gene naturally occurring in said organism. In some instances, the organism is naturally photosynthetic. In other instances, the additional genes encode enzymes in the isoprenoid pathway, MVA pathway, or MEP pathway. In still another embodiment, the present disclosure provides a method for transforming a cell or organism comprising inserting into said cell or organism a chloroplast and a vector comprising all essential chloroplast genes.

The present disclosure also provides a method of producing an artificial chloroplast genome comprising the steps of: (a) providing a vector comprising one or more essential chloroplast genes; (b) adding to said vector a DNA fragment; (c) transforming a cell or organism with the vector produced by step (b); and (d) determining whether chloroplast function exists with said added DNA fragment. In some instances, the added DNA fragments comprises one or more coding regions for an enzyme in the isoprenoid, MVA or MEP pathway.

The present disclosure also provides a shuttle vector comprising at least about 85%, at least about 87%, at least about 90%, at least about 92%, or at least about 95% of a chloroplast genome. The genome may be modified by the insertion, deletion and/or rearrangement of one or more nucleotide sequences. Also provided herein is a vector comprising an isolated, functional chloroplast genome. A chloroplast genome useful in such a vector may be modified by the insertion, deletion and/or rearrangement of one or more nucleotide sequences.

Further provided herein is a method of producing an artificial chloroplast genome comprising the steps of: (a) providing a vector comprising all essential chloroplast genes; and (b) removing, adding, mutating, or rearranging DNA from the chloroplast genome. Such a method may further comprise the steps of transforming a redacted genome into a host organism; and (d) determining chloroplast function in the host organism. In some instances, steps (b), (c), and (d) are repeated. In still other instances, the chloroplast genome is from an organism selected from the group consisting of: soybeans, tomatoes, potatoes, wheat, rice, corn, barley, oats, rye, cotton, Arabidopsis, tobacco, and legumes. In other instances, the host organism is selected from the group consisting of: soybeans, tomatoes, potatoes, wheat, rice, corn, barley, oats, rye, cotton, Arabidopsis, tobacco, and legumes. For some embodiments, the method may further comprise the step of removing redundant DNA from a chloroplast genome. In other embodiments, the vector comprises all or substantially all of a chloroplast genome. A chloroplast genome useful in the present disclosure may be cloned from a photosynthetic organism or may be a synthetic chloroplast genome. In some instances, the vector further comprises a gene not naturally occurring in the host organism, for example a gene from the isoprenoid pathway, MVA pathway, or MEP pathway.

Yet another method provided herein is a method of producing an artificial chloroplast genome comprising the steps of: (a) providing a vector comprising an entire chloroplast genome; (b) deleting a portion of said entire chloroplast genome; and (c) determining whether chloroplast function exists without said deleted portion. In another aspect of the present invention, a composition comprising an isolated and functional chloroplast genome is provided. In some instances, a composition comprises a modification to said chloroplast genome.

Further provided herein is an ex vivo vector comprising a nucleic acid comprising at least about 85%, at least about 87%, at least about 90%, at least about 92%, or at least about 95% of a chloroplast genome and a manipulation in one or more nucleic acids in the vector. In some instances, the nucleic acid is cloned from a photosynthetic vascular plant such as soybeans, tomatoes, potatoes, wheat, rice, corn, barley, oats, rye, cotton, Arabidopsis, tobacco, and legumes. In some instances, the nucleic acid is synthetic. A vector may further comprise an expression cassette and an expression cassette may further comprise a region for integration into target DNA. In some instances, the target DNA is organelle DNA. A vector useful in the present invention may further comprise one or more selection markers, for example an auxotrophic marker, an antibiotic resistance marker, a chloroplast marker, or combinations thereof. In some embodiments, a manipulation in one or more nucleic acids in a vector may be an addition, deletion, mutation, or rearrangement. Expression of the vector may result in production of a product not naturally produced by a host cell and/or an increase production of a product naturally produced by a host cell. Examples of some products of the present invention include a terpene, terpenoid, fatty acid, biomass degrading enzyme, therapeutic protein and/or a phytosterol.

Also provided herein is an ex vivo vector comprising a nucleic acid comprising at least about 135 kilobases or at least about 150 kilobases of a chloroplast genome and a manipulation in one or more nucleic acids in said vector. In some instances, the nucleic acid is cloned from a photosynthetic vascular plant such as soybeans, tomatoes, potatoes, wheat, rice, corn, barley, oats, rye, cotton, Arabidopsis, tobacco, and legumes. In some instances, the nucleic acid is synthetic. A vector of the present disclosure may further comprise an expression cassette and an expression cassette may further comprise a region for integration into target DNA. In some instances, the target DNA is organelle DNA. A vector useful in the present disclosure may further comprise one or more selection markers, for example an auxotrophic marker, an antibiotic resistance marker, a chloroplast marker, or combinations thereof. In some embodiments, a manipulation in one or more nucleic acids in a vector may be an addition, deletion, mutation, or rearrangement. Expression of the vector may result in production of a product not naturally produced by a host cell and/or an increase production of a product naturally produced by a host cell. Examples of some products of the present invention include a terpene, terpenoid, fatty acid, a biomass degrading enzyme, a therapeutic protein and/or a phytosterol.

Further provided herein is a method of producing a vector containing a reconstructed genome, comprising: introducing two or more vectors into a host cell, wherein the vectors comprise fragments of a genome, recombining the vectors into a single vector comprising at least about 85%, at least about 87%, at least about 90%, at least about 92%, or at least about 95% of a genome, thereby producing a vector containing a reconstructed genome. In some instances, the host cell is eukaryotic, for example, a yeast such as S. cerevisiae. In other instances, the genome is an organelle genome. The organelle may be a chloroplast, for example a chloroplast from a vascular plant, particularly a plant such as soybeans, tomatoes, potatoes, wheat, rice, corn, barley, oats, rye, cotton, Arabidopsis, tobacco, and legumes. In some instances, the two or more vectors comprise a selectable marker. In other instances, at least one of said fragments is synthetic. In still other instances, a further step comprising modifying a portion of the genome is useful in this method. Such a modification may comprise an addition, deletion, mutation, or rearrangement. In other embodiments, the modification is the addition of an exogenous nucleic acid which results in the production or increased production of a terpene, terpenoid, fatty acid, a biomass degrading enzyme, a therapeutic protein and/or a phytosterol.

Large DNA Cloning and Content

An advantage of this invention is that it provides for the cloning, manipulation, and delivery of a vector containing chloroplast genome DNA consisting of up to all chloroplast genes (or sequences). The chloroplast genome DNA contained in the vector can be obtained by recombination of a hybrid cloning vector with one contiguous fragment of DNA or by recombination of two or more contiguous fragments of DNA.

The methods and compositions disclosed herein may include captured and/or modified large pieces of DNA may comprise DNA from an organelle, such as mitochondrial DNA or plastid DNA (e.g., chloroplast DNA). The captured and/or modified large pieces of DNA may also comprise the entirety of an organelle's genome, e.g., a chloroplast genome. In other embodiments, the captured and/or modified large pieces of DNA comprise a portion of a chloroplast genome, for example 85%, 87%, 90%, 92%, 95% or more. A chloroplast genome may originate from any vascular or non-vascular plant, including algae, bryophytes (e.g., mosses, ferns), gymnosperms (e.g., conifers), and angiosperms (e.g., flowering plants—trees, grasses, herbs, shrubs). A chloroplast genome, or essential portions thereof, may comprise synthetic DNA, rearranged DNA, deletions, additions, and/or mutations. A chloroplast genome, or portions thereof, may comprise one or more deletions, additions, mutations, and/or rearrangements. The deletions, additions, mutations, and/or rearrangements may be naturally found in an organism, for example a naturally occurring mutation, or may not be naturally found in nature. The chloroplast or plastid genomes of a number of organisms are widely available, for example, at the public database from the NCBI Organelle Genomes section available at http://www.ncbi.nlm.nih.gov/genomes/static/euk_o.html.

The target DNA sequence described herein may comprise 1, 2, 3, 4, 5, 6, 7, 8, 9, 10 or more deletions, additions, mutations, and/or rearrangements as compared to a control sequence (naturally occurring sequence). Alternatively, the target DNA sequence described herein may comprise up to 1, 2, 3, 4, 5, 6, 7, 8, 9, or 10 deletions, additions, mutations, and/or rearrangements as compared to a control sequence. In some embodiments, the mutations may be functional or nonfunctional. For example, a functional mutation may have an effect on a cellular function when the mutation is present in a host cell as compared to a control cell without the mutation. A non-functional mutation may be silent in function, for example, there is no discernable difference in phenotype of a host cell without the mutation as compared to a cell with the mutation.

Captured and/or modified large pieces of DNA (e.g., target DNA), may comprise a minimal or minimized chloroplast genome (e.g., the minimum number of genes and/or DNA fragment, required for chloroplast functionality). The captured and/or modified DNA may comprise the essential chloroplast genes, it may comprise a portion or all, or substantially all of the essential chloroplast genes. An essential gene may be a gene that is essential for one or more metabolic processes or biochemical pathways. An essential gene may be a gene required for chloroplast function, such as photosynthesis, carbon fixation, or hydrocarbon production. An essential gene may also be a gene that is essential for gene expression, such as transcription, translation, or other process(es) that affect gene expression. The essential genes may comprise mutations or rearrangements. Essential genes may also comprise a minimally functional set of genes to perform a function. For example, a particular function (e.g., photosynthesis) may be performed inefficiently by a set of genes/gene products, however, this set would still comprise essential genes because the function is still performed. Thus, in one embodiment, substantially all essential chloroplast genes comprises the collection of gene needed to render the organism photoautotrophic.

Modified DNA may comprise at least 5, 10, 15, 20, 25, 30, 40, or 50 essential genes. Modified DNA may also comprise between 5 and 10, between 10 and 15, between 15 and 20, between 20 and 25, between 25 and 30, between 30 and 40, or between 40 and 50 essential genes. In some embodiments, the DNA may comprise essential chloroplast genomic sequence of up to about 135 kb or up to about 150 kb in length. The DNA may comprise essential chloroplast genes as well as non-essential chloroplast gene sequences. The DNA may be single stranded or double stranded, linear or circular, relaxed or supercoiled. The DNA may also be in the form of an expression cassette. For example, an expression cassette may comprise an essential gene to be expressed in a host cell. The expression cassette may comprise one or more essential genes as well as DNA sequences that promote the expression of the essential genes. The expression cassette may also comprise a region for integration into target DNA of a host. The expression cassette may also comprise one or more essential genes and one or more genes not naturally occurring in a host cell comprising the expression cassette. One of ordinary skill in the arts will easily ascertain various combinations of the aforementioned aspects of the expression cassettes.

In other instances, captured and/or modified pieces of DNA may comprise the entire genome of a plastid or organelle. For example, about 10%, 11%, 12%, 13%, 14%, 15%, 16%, 17%, 18%, 19%, 20%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of a plastid genome. In one embodiment the captured and/or modified large pieces of DNA may comprise 10-100%, 20-100%, 30-100%, 40-100%, 50-100%, 60-100%, 70-100%, 80-100%, or 90-100% of the entire genome of a plastid or cell.

In still other instances, the captured and/or modified large pieces of DNA may comprise about 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb, 36 kb, 37 kb, 38 kb, 39 kb, 40 kb, 41 kb, 42 kb, 43 kb, 44 kb, 45 kb, 46 kb, 47 kb, 48 kb, 49 kb, 50 kb, 51 kb, 52 kb, 53 kb, 54 kb, 55 kb, 56 kb, 57 kb, 58 kb, 59 kb, 60 kb, 61 kb, 62 kb, 63 kb, 64 kb, 65 kb, 66 kb, 67 kb, 68 kb, 69 kb, 70 kb, 71 kb, 72 kb, 73 kb, 74 kb, 75 kb, 76 kb, 77 kb, 78 kb, 79 kb, 80 kb, 81 kb, 82 kb, 83 kb, 84 kb, 85 kb, 86 kb, 87 kb, 88 kb, 89 kb, 90 kb, 91 kb, 92 kb, 93 kb, 94 kb, 95 kb, 96 kb, 97 kb, 98 kb, 99 kb, 100 kb, 101 kb, 102 kb, 103 kb, 104 kb, 105 kb, 106 kb, 107 kb, 108 kb, 109 kb, 110 kb, 111 kb, 112 kb, 113 kb, 114 kb, 115 kb, 116 kb, 117 kb, 118 kb, 119 kb, 120 kb, 121 kb, 122 kb, 123 kb, 124 kb, 125 kb, 126 kb, 127 kb, 128 kb, 129 kb, 130 kb, 131 kb, 132 kb, 133 kb, 134 kb, 135 kb, 136 kb, 137 kb, 138 kb, 139 kb, 140 kb, 141 kb, 142 kb, 143 kb, 144 kb, 145 kb, 146 kb, 147 kb, 148 kb, 149 kb, 150 kb, 151 kb, 152 kb, 153 kb, 154 kb, 155 kb, 156 kb, 157 kb, 158 kb, 159 kb, 160 kb, 161 kb, 162 kb, 163 kb, 164 kb, 165 kb, 166 kb, 167 kb, 168 kb, 169 kb, 170 kb, 171 kb, 172 kb, 173 kb, 174 kb, 175 kb, 176 kb, 177 kb, 178 kb, 179 kb, 180 kb, 181 kb, 182 kb, 183 kb, 184 kb, 185 kb, 186 kb, 187 kb, 188 kb, 189 kb, 190 kb, 191 kb, 192 kb, 193 kb, 194 kb, 195 kb, 196 kb, 197 kb, 198 kb, 199 kb, 200 kb, 201 kb, 202 kb, 203 kb, 204 kb, 205 kb, 206 kb, 207 kb, 208 kb, 209 kb, 210 kb, 211 kb, 212 kb, 213 kb, 214 kb, 215 kb, 216 kb, 217 kb, 218 kb, 219 kb, 220 kb, 221 kb, 222 kb, 223 kb, 224 kb, 225 kb, 226 kb, 227 kb, 228 kb, 229 kb, 230 kb, 231 kb, 232 kb, 233 kb, 234 kb, 235 kb, 236 kb, 237 kb, 238 kb, 239 kb, 240 kb, 241 kb, 242 kb, 243 kb, 244 kb, 245 kb, 246 kb, 247 kb, 248 kb, 249 kb, 50 kb, 51 kb, 252 kb, 253 kb, 254 kb, 255 kb, 256 kb, 257 kb, 258 kb, 259 kb, 260 kb, 261 kb, 262 kb, 263 kb, 264 kb, 265 kb, 266 kb, 267 kb, 268 kb, 269 kb, 270 kb, 271 kb, 272 kb, 273 kb, 274 kb, 275 kb, 276 kb, 277 kb, 278 kb, 279 kb, 280 kb, 281 kb, 282 kb, 283 kb, 284 kb, 285 kb, 286 kb, 287 kb, 288 kb, 289 kb, 290 kb, 291 kb, 292 kb, 293 kb, 294 kb, 295 kb, 296 kb, 297 kb, 298 kb, 299 kb, 300 kb, 301 kb, 302 kb, 303 kb, 304 kb, 305 kb, 306 kb, 307 kb, 308 kb, 309 kb, 310 kb, 311 kb, 312 kb, 313 kb, 314 kb, 315 kb, 316 kb, 317 kb, 318 kb, 319 kb, 320 kb, 321 kb, 322 kb, 323 kb, 324 kb, 325 kb, 326 kb, 327 kb, 328 kb, 329 kb, 330 kb, 331 kb, 332 kb, 333 kb, 334 kb, 335 kb, 336 kb, 337 kb, 338 kb, 339 kb, 340 kb, 341 kb, 342 kb, 343 kb, 344 kb, 345 kb, 346 kb, 347 kb, 348 kb, 349 kb, 350 kb, 351 kb, 352 kb, 353 kb, 354 kb, 355 kb, 356 kb, 357 kb, 358 kb, 359 kb, 360 kb, 361 kb, 362 kb, 363 kb, 364 kb, 365 kb, 366 kb, 367 kb, 368 kb, 369 kb, 370 kb, 371 kb, 372 kb, 373 kb, 374 kb, 375 kb, 376 kb, 377 kb, 378 kb, 379 kb, 380 kb, 381 kb, 382 kb, 383 kb, 384 kb, 385 kb, 386 kb, 387 kb, 388 kb, 389 kb, 390 kb, 391 kb, 392 kb, 393 kb, 394 kb, 395 kb, 396 kb, 397 kb, 398 kb, 399 kb, 400 kb or more genomic (e.g., nuclear or organelle) DNA. In some embodiments the captured and or modified large pieces of DNA may comprise about 10-400 kb, 50-350 kb, 100-300 kb, 100-200 kb, 200-300 kb, 150-200 kb, 200-250 kb genomic DNA

In still other instances, the captured and or modified large pieces of DNA may comprise about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 50, 51, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or more open reading frames, partial open reading frames, pseudogenes and/or repeating sequences.

The present disclosure also provides vectors comprising a cassette-able chloroplast genome or portion thereof (e.g., a removable DNA fragment comprising a chloroplast genome or functional portion thereof). A vector of the present invention may comprise functional chloroplast units (e.g., a unit essential for metabolic processes, photosynthesis, gene expression, photosystem I, photosystem II). Vectors of the present invention may comprise a transplantable chloroplast genome or portion thereof. Additionally, the vectors of the present invention may comprise a transferable chloroplast genome or portion thereof. In other embodiments, the vectors comprise: 1) one or more large pieces of modified DNA; 2) all genes necessary to carry out photosynthesis; 3) all genes required for chloroplast survival and/or function; 4) essential chloroplast genes; and/or 5) sufficient naturally occurring or modified chloroplast genes to perform one or more chloroplast functions, such as photosynthesis. A vector may comprise a portion, substantially all, or all of the essential chloroplast genes. A vector may comprise at least 5, 10, 15, 20, 25, 30, 40, 50, 60, 70, 80, 90, 100 or more essential genes.

A vector may comprise chloroplast DNA of 10, 20, 30, 40, 50, 60, 70, 80, 90, 100, 110, 120, 130, 140, 150, 160, 170, 180, 190, 200, 210, 220, 230, 240, 250 kb or more in length. A vector may comprise essential chloroplast genes as well as non-essential chloroplast gene sequences. A vector may comprise one or more, or all, essential chloroplast genes and/or one or more genes not naturally occurring in a host cell comprising a vector. In some embodiments, a vector may comprise chloroplast genes and genes not naturally occurring in the chloroplast. A vector may comprise one or more essential chloroplast genes and/or one or more DNA sequences or genes involved in chloroplast function, photosynthesis, carbon fixation, and/or hydrocarbon production. For example, a vector may comprise a sequence required for photosynthesis and a sequence involved in the isoprenoid production, MVA, and/or MEP pathways, such as a DNA sequence encoding a terpene synthase, or other polypeptide that produces a hydrocarbon, such as a terpene or isoprenoid. The invention further provides methods for cloning, manipulating and delivering a large target nucleic acid to a cell or particle, such as, for example, yeasts or bacteria. Certain embodiments make use of a hybrid yeast-bacteria cloning system (See, e.g., U.S. Pat. Nos. 5,866,404 and 7,083,971 and Hokanson et al., (2003)Human Gene Ther.: 14: 329-339). The vectors herein (e.g., cloning system) are comprised of a shuttle vector that contains elements for function and replication in both yeast and bacteria, allowing them to stably function and replicate in either organism. This composition of functional and replicative elements yields a hybrid system which enjoys the benefits of both genetic engineering systems. The genetics of yeast (e.g., S. cerevisiae) are well understood and a powerful assortment of molecular biology tools exists for genetic engineering in bacteria.

Another aspect produces a gap-filled vector by homologous recombination among the two arms and the target nucleic acid. In still another embodiment, at least one arm further comprises an origin of replication. In another embodiment, each arm further comprises a rare restriction endonuclease recognition site. Homologous recombination may be performed in vitro or in vivo, for example, in a fungal cell (e.g., S. cerevisiae, Sz. pombe or U. maydis). Also provided is a eukaryotic host cell harboring the recombinational cloning system or vector according to the present disclosure, for example, in a fungal cell (e.g., S. cerevisiae, Sz. pombe or U. maydis).

A gap-filled linear vector may be converted to a circular vector in vitro (e.g. using T4 ligase) or in vivo, for example, in a bacterium. The circular vectors of interest can be amplified, purified, cut and used to recover sufficient amounts of DNA to be introduced either directly into a cell or into a suitable delivery system for subsequent delivery to a target cell. The methodology offers great versatility to clone and to modify any large bacterial or non-bacterial genome, and easily facilitate the use thereof as recombinational vectors. Direct delivery of a gap-filled vector into a cell may be performed by methods well known in the field such as, for example, calcium phosphate transformation methodologies or electroporation (see Sambrook et al., supra).

Accordingly, provided herein is a method for producing a recombinant delivery unit including the steps of: (a) producing a gap-filled vector containing a target sequence; (b) optionally circularizing the gap-filled vector segments of the invention; (c) propagating the vector; and (d) introducing the gap-filled vector in a delivery unit.

Bacterial systems are useful for amplifying and purifying DNA, and for functionally testing the genetic modifications and their effect on pathways. One embodiment of the present invention provides a cloning system that will aid in the cloning and modifying of any large genome and easily facilitate the cloning and introduction of pathways. With the ability to deliver whole pathways, certain embodiments of the present invention allow for a system biological approach to problem solving.

In general, target DNA (e.g., genomic DNA) may be captured by creating sites allowing for homologous recombination in the vector. For example, such sites may be created by, but not limited to, gap-repair cloning, wherein a gap is created in the vector, usually by restrictive enzyme digestion prior to transformation into the yeast. When the target DNA is mixed with the vector, the target DNA is recombined into the vector. This operation is called “gap filling.” This recombination can occur in bacteria, yeast, the original host organism, another organism, or in vitro. In some embodiments, recombination is performed in yeast because of the high rate of homologous recombination. Once captured, the target DNA can be modified in many ways including adding, altering, or removing DNA sequences. In some embodiments, the target DNA is genomic DNA. In other embodiments, the target DNA is organelle (e.g., mitochondria or chloroplast) DNA.

In some embodiments, target DNA is modified by adding, altering or removing genes, coding sequences, partial coding sequences, regulatory elements, positive and/or negative selection markers, recombination sites, restriction sites, and/or codon bias sites. For example, the target DNA sequence may be codon biased for expression in the organism being transformed. The skilled artisan is well aware of the “codon-bias” exhibited by a specific host cell or organelle in usage of nucleotide codons to specify a given amino acid. Without being bound by theory, by using preferred codons, the rate of translation may be greater. Therefore, when synthesizing a gene for improved expression, it may be desirable to design the gene such that its frequency of codon usage approaches the frequency of preferred codon usage. The codons of the present invention may be A/T rich, for example, A/T rich in the third nucleotide position of the codons. In some embodiments, at least 50% of the third nucleotide position of the codons are A or T. In other embodiments, at least 60%, 70%, 80%, 90%, or 99% of the third nucleotide position of the codons are A or T. (see also U.S. Publication No. 2004/0014174 and U.S. Pat. No. 5,545,817).

Such manipulations are well known in the art and can be performed in numerous ways. In some embodiments, the modifications may be performed using cloned sequences. In other embodiments, the modifications may be performed using synthetic DNA.

Genetic manipulations include cloning large pieces of target DNA (e.g., chromosomes, genomes) and/or dividing and reorganizing target DNA based on functional relations between genes, such as metabolic pathways or operons. Genetic manipulations also include introducing and removing metabolic pathways, recombining DNA into functional units (e.g., metabolic pathways, synthetic operons), and/or determining sites of instability in large pieces of DNA (e.g., sites where a native or non-native host tends to delete or recombine a sequence of DNA).

Target DNA may be DNA from a prokaryote. Target DNA may also be genomic DNA, mitochondrial DNA, or chloroplast DNA from a eukaryote. Examples of such organisms from which genomic and/or organelle DNA may serve as target DNA include, but are not limited to vascular plants and more specifically soybeans, tomatoes, potatoes, wheat, rice, corn, barley, oats, rye, cotton, Arabidopsis, tobacco, and legumes. One of skill in the art will recognize that these organisms are listed only as examples and that the methods disclosed herein are applicable to the large DNA from any organism, including bacteria, plants, fungi, protists, and animals. Genetic manipulations of the present invention may include stabilizing large pieces of DNA by removing or inserting sequences that force transformed cells to preserve certain sequences of DNA and to stably maintain the sequences in its progeny. Genetic manipulations may also include altering codons of the target DNA, vector DNA, and/or synthetic DNA to reflect any codon bias of the host organism. Additionally, genetic manipulations may include determining the minimal set of genes required for an organism to be viable. In another embodiment, the genetic manipulations include determining the minimal set of genes required for a certain metabolic pathway to be created or maintained.

The genetic manipulations of the present disclosure may include determining redundant genes both within a genome, and between two genomes (e.g., redundancy between the nuclear and chloroplast genome). Additionally, the genetic manipulations may include determining a functional sequence of DNA that could be artificially synthesized (e.g. the genes in a certain metabolic pathway, the genes of a functional genome). In another embodiment, the genetic manipulations include creating DNA and genomes packaged into cassettes (e.g., sites within a vector where genes can be easily inserted or removed). The genetic manipulations may also include creating a nuclear or organelle genome that is viable in multiple species (e.g. a transplantable chloroplast genome). Furthermore, the genetic manipulations may include a method for testing the viability of any of these manipulations or creations (e.g., transferring a shuttle vector back into a host system and assaying for survival).

Vectors, Markers and Transformation

A vector or other recombinant nucleic acid molecule may include a nucleotide sequence encoding a selectable marker. The term or “selectable marker” or “selection marker” refers to a polynucleotide (or encoded polypeptide) that confers a detectable phenotype. A selectable marker generally encodes a detectable polypeptide, for example, a green fluorescent protein or an enzyme such as luciferase, which, when contacted with an appropriate agent (a particular, wavelength of light or luciferin, respectively) generates a signal that can be detected by eye or using appropriate instrumentation (Giacomin, Plant Sci. 116:59-72, 1996; Scikantha, J. Bacteriol. 178:121, 1996; Gerdes, FEBS Lett. 389:44-47, 1996; see, also, Jefferson, EMBO J. 6:3901-3907, 1997, fl-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain prokaryotic cells, yeast cell, plant cells or any combination that express the marker and, therefore, can be useful as a component of a vector of the present disclosure (see, for example, Bock, supra, 2001). Examples of selectable markers include, but are not limited to, those that confer antimetabolite resistance, for example, dihydrofolate reductase, which confers resistance to methotrexate (Reiss, Plant Physiol. (Life Sci. Adv.) 13:143-149, 1994); neomycin phosphotransferase, which confers resistance to the aminoglycosides neomycin, kanamycin and paromycin (Herrera-Estrella, EMBO J. 2:987-995, 1983), hygro, which confers resistance to hygromycin (Marsh, Gene 32:481-485, 1984), trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (White et al., Nucl. Acids Res. 18:1062, 1990; Spencer et al., Theor. Appl. Genet. 79:625-631, 1990), a mutant EPSPV-synthase, which confers glyphosate resistance (Hinchee et al., BioTechnology 91:915-922, 1998), a mutant acetolactate synthase, which confers imidazolione or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants (see, for example, Maliga et al., Methods in Plant Molecular Biology, Cold Spring Harbor Laboratory Press, 1995, page 39).

Methods for nuclear and plastid transformation are routine and well known for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

Microprojectile mediated transformation also can be used to introduce a polynucleotide into a plant cell chloroplast (Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PDS-1000 particle gun (BioRad; Hercules Calif.). Methods for the transformation using biolistic methods are well known in the art (see, e.g.; Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (Duan et al., Nature Biotech. 14:494-498, 1996; Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, the glass bead agitation method, and the like.

Transformation frequency may be increased by replacement of recessive rRNA or r-protein antibiotic resistance genes with a dominant selectable marker, including, but not limited to the bacterial aadA gene (Svab and Maliga, Proc. Natl. Acad. Sci., USA 90:913-917, 1993). Approximately 15 to 20 cell division cycles following transformation are generally required to reach a homoplastidic state. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

Any of the nucleotide sequences of target DNA, vector DNA, or synthetic DNA in the vectors disclosed herein can further include codons biased for expression of the nucleotide sequences in the organism transformed. In some instances, codons in the nucleotide sequences are A/T rich in a third nucleotide position of the codons. For example, at least 50% of the third nucleotide position of the codons may be A or T. In other instances, the codons are G/C rich, for example at least 50% of the third nucleotide positions of the codons may be G or C.

The nucleotide sequences of the shuttle vectors of the present disclosure can be adapted for chloroplast expression. For example, a nucleotide sequence herein can comprise a chloroplast specific promoter or chloroplast specific regulatory control region.

In embodiments where a vector encodes genes capable of fuel production, fuel products are produced by altering the enzymatic content of the cell to increase the biosynthesis of specific fuel molecules. For example, nucleotides sequences (e.g., an ORF isolated from an exogenous source) encoding biosynthetic enzymes can be introduced into the chloroplast of a photosynthetic organism. Nucleotide sequences encoding fuel biosynthetic enzymes can also be introduced into the nuclear genome of the photosynthetic organisms. Nucleotide sequences introduced into the nuclear genome can direct accumulation of the biosynthetic enzyme in the cytoplasm of the cell, or may direct accumulation of the biosynthetic enzyme in the chloroplast of the photosynthetic organism.

Any of the nucleotide sequences herein may further comprise a regulatory control sequence. Regulatory control sequences can include one or more of the following: a promoter, an intron, an exon, processing elements, 3′ untranslated region, 5′ untranslated region, RNA stability elements, or translational enhancers. A promoter may be one or more of the following: a promoter adapted for expression in the organism, an algal promoter, a chloroplast promoter, and a nuclear promoter, any of which may be a native or synthetic promoters. A regulatory control sequence can be inducible or autoregulatable. A regulatory control sequence can include autologous and/or heterologous sequences. In some cases, control sequences can be flanked by a first homologous sequence and a second homologous sequence. The first and second homologous sequences can each be at least 500 nucleotides in length. The homologous sequences can allow for either homologous recombination or can act to insulate the heterologous sequence to facilitate gene expression.

Vectors may also comprise sequences involved in producing products useful as biopharmaceuticals, such as, but not limited to, antibodies (including functional portions thereof), interleukins and other immune modulators, and antibiotics. See, e.g., Mayfield et al., (2003) Proc. Nat'l Acad. Sci.: 100 (438-42) and U.S. Pub. No. 2004/0014174.

Vectors may comprise a cassette-able bacterial genome or portion thereof (e.g., a removable DNA fragment comprising a bacterial genome or functional portion thereof). Additionally, vectors may comprise functional genomic units (e.g., a unit essential for metabolic processes, biochemical pathways, gene expression). Vectors may also comprise a transplantable bacterial genome or portion thereof. Vectors may comprise a transferable bacterial genome or portion thereof.

In some embodiments, the large piece of target DNA is modified. The modified DNA may comprise all genes necessary to carry out ethanologenesis, all genes required for the Entner-Duodorff pathway, the glucose tolerance pathway, the ethanol tolerance pathway, the carboxylic acid byproduct resistance pathway, the acetic acid tolerance pathway, the sugar transport pathway, sugar fermentation pathways, and/or the cellulose and hemicellulose digestive pathways.

Hybrid cloning systems and methods of the invention combine the high versatility of yeast as a system for the capture and manipulation of a given nucleic acid and the high efficiency of bacterial systems for the amplification of such nucleic acid. Recombinational vectors relying on homologous recombination to mediate the isolation, manipulation and delivery of large nucleic acid fragments were constructed. Also described herein are methods for using such recombinational cloning vectors to clone, to manipulate and to deliver large nucleic acids. Additionally, this disclosure provides methods for using such recombinational cloning systems as potentiators of biochemical pathway analysis, organelle analysis, and synthetic chloroplast construction.

The vectors herein may be introduced into yeast. The yeast may be a suitable strain of Saccharomyces cerevisiae; however, other yeast models may be utilized. Introduction of vectors into yeast may allow for genetic manipulation of the vectors. Yeast vectors have been described extensively in the literature and methods of manipulating the same also are well known as discussed hereinafter (see e.g., Ketner et al. (1994) Proc. Natl. Acad. Sci. (USA) 91:6186 6190).

Following genetic manipulation, the present system allows for the transition to a bacterial environment, suitable for the preparation of larger quantities of nucleic acids. Also provided is a shuttle vector comprising a yeast selectable marker, a bacterial selectable marker, a telomere, a centromere, a yeast origin of replication, and/or a bacterial origin of replication.

Shuttle vectors described herein may enable homologous recombination in yeast to capture and to integrate in a vector of interest a target nucleic acid of interest. Shuttle vectors may allow for the manipulation of target DNA in any of the hosts to which the vectors can be introduced. In some embodiments, after desired manipulations, shuttle vector components may be removed, leaving just the modified target DNA. Such extraction of vector sequences can be performed using standard methodologies and may occur in any host cell. The target nucleic acid of interest can be a large nucleic acid, and can include, for example, a vector, such as a viral vector, including the foreign gene of interest contained therein. The target nucleic acid can also be a bacterial (including archaebacteria and eubacteria), viral, fungal, protist, plant or animal genome, or a portion thereof. For example, a target nucleic acid of the present disclosure may comprise the chloroplast genome of a eukaryotic organism.

Shuttle vectors according to the disclosure may comprise an appropriately oriented DNA that functions as a telomere in yeast and a centromere. Any suitable telomere may be used. Suitable telomeres include without limitation telomeric repeats from many organisms, which can provide telomeric function in yeast. The terminal repeat sequence in humans (TTAGGG)_{N}, is identical to that in trypanosomes and similar to that in yeast ((TG)₁₋₃)_{N} and Tetrahymena (TTGGG)_{N} (Szostak & Blackburn (1982) Cell 29:245 255; Brown (1988) EMBO J. 7:2377 2385; and Moyzis et al. (1988) Proc. Natl. Acad. Sci. 85:6622 6626).

The term “centromere” is used herein to identify a nucleic acid, which mediates the stable replication and precise partitioning of the vectors at meiosis and at mitosis thereby ensuring proper segregation into daughter cells. Suitable centromeres include, without limitation, the yeast centromere, CEN4, which confers mitotic and meiotic stability on large linear plasmids (Murray & Szostak (1983) Nature 305:189 193; Carbon (1984) Cell 37:351 353; and Clark et al. (1990) Nature 287:504 509)).

In some embodiments, at least one of the two segments of the circular vector according to the disclosure includes at least one replication system that is functional in a host cell/particle of choice. As it will become apparent hereinafter, one of skill will realize that the manipulation, amplification and/or delivery of a target nucleic acid of choice may entail the use of more than one host cell/particle. Accordingly, more than one replication system functional in each host cell/particle of choice may be included.

When a host cell(s) is a prokaryote, particularly E. coli, replication system(s) include those which are functional in prokaryotes, such as, for example, P1 plasmid replicon, ori, P1 lytic replicon, ColE1, BAC, single copy plasmid F factors and the like. Either one or both segments, and/or the circular vector, may further include a yeast origin of replication capable of supporting the replication of large nucleic acids. Non-limiting examples of replication regions according to the invention include the autonomously replicating sequence or “ARS element.” ARS elements were identified as yeast sequences that conferred high-frequency transformation. Tetrahymena DNA termini have been used as ARS elements in yeast along with ARS1 and ARS4 (Kiss et al. (1981) Mol. Cell. Bol. 1:535 543; Stinchcomb et al. (1979) Nature 282:39; and Barton & Smith (1986) Mol. Cell. Biol. 6:2354). For each segment (e.g., those corresponding to the yeast and bacterial elements of the gap-filling shuttle vector) there may be two or more origins of replication.

The first and/or the second segment according to an aspect of the disclosure may be joined in a circularized vector form (e.g., plasmid form). Circularization can occur in vivo or in vitro using the segment of interest. Alternatively, a circular vector of interest can be used. As used herein, the term “vector” designates a plasmid or phage DNA or other nucleic acid into which DNA or other nucleic acid may be cloned. The vector may replicate autonomously in a host cell and may be characterized further by one or a small number of restriction endonuclease recognition sites at which such nucleic acids may be cut in a determinable fashion and into which nucleic acid fragments may be inserted. The vector further may contain one or more selectable markers suitable for the identification of cells transformed with the vector.

Target nucleic acids of the invention may vary considerably in complexity. The target nucleic acid may include viral, prokaryotic or eukaryotic DNA, cDNA, exonic (coding), and/or intronic (noncoding) sequences. Hence, the target nucleic acid may include one or more genes. A target nucleic acid may be a chromosome, genome, or operon and/or a portion of a chromosome, genome or operon. A target nucleic acid may comprise coding sequences for all the genes in a pathway, the minimum complement of genes necessary for survival of an organelle, and/or the minimum complement of genes necessary for survival of an organism. A target nucleic acid may comprise Zymomonas mobilis DNA sequence, including, but not limited to genomic DNA and/or cDNA. A target nucleic acid may comprise eukaryotic chloroplast DNA sequence, including but not limited to, chloroplast genome DNA and/or cDNA. A target nucleic acid may comprise cyanobacteria DNA, including but not limited to genomic DNA and/or cDNA. The target nucleic acid also may be of any origin and/or nature.

It may be desirable for the gene to also comprise a promoter operably linked to the coding sequence in order to effectively promote transcription. Enhancers, repressors and other regulatory sequences may also be included in order to modulate activity of the gene, as is well known in the art. A gene as provided herein can refer to a gene that is found in the genome of the individual host cell (i.e., endogenous) or to a gene that is not found in the genome of the individual host cell (i.e., exogenous or a “foreign gene”). Foreign genes may be from the same species as the host or from different species. For transfection of a cell using DNA containing a gene with the intent that the gene will be expressed in the cell, the DNA may contain any control sequences necessary for expression of the gene in the required orientation for expression. The term “intron” as used herein, refers to a DNA sequence present in a given gene which is not translated into protein and is generally found between exons.

Genetic elements, or polynucleotides comprising a region that encodes a polypeptide or a region that regulates transcription or translation or other processes important to expression of the polypeptide in a host cell, or a polynucleotide comprising both a region that encodes a polypeptide and a region operably linked thereto that regulates expression. Genetic elements may be comprised within a vector that replicates as an episomal element; that is, as a molecule physically independent of the host cell genome. They may be comprised within mini-chromosomes, such as those that arise during amplification of transfected DNA by methotrexate selection in eukaryotic cells. Genetic elements also may be comprised within a host cell genome; not in their natural state but, rather, following manipulation such as isolation, cloning and introduction into a host cell in the form of purified DNA or in a vector, among others.

Vectors of the present disclosure may contain sufficient linear identity or similarity (homology) to have the ability to hybridize to a portion of a target nucleic acid made or which is single-stranded, such as a gene, a transcriptional control element or intragenic DNA. Without being bound to theory, such hybridization is ordinarily the result of base-specific hydrogen bonding between complementary strands, preferably to form Watson-Crick base pairs. As a practical matter, such homology can be inferred from the observation of a homologous recombination event. In some embodiments, such homology is from about 8 to about 1000 bases of the linear nucleic acid. In other embodiments, such homology is from about 12 to about 500 bases. One skilled in the art will appreciate that homology may extend over longer stretches of nucleic acids.

Homologous recombination is a type of genetic recombination, a process of physical rearrangement occurring between two strands of DNA. Homologous recombination involves the alignment of similar sequences, a crossover between the aligned DNA strands, and breaking and repair of the DNA to produce an exchange of material between the strands. The process homologous recombination naturally occurs in organisms and is also utilized as a molecular biology technique for introducing genetic changes into organism.

The vectors disclosed herein may be modified further to include functional entities other than the target sequence which may find use in the preparation of the construct(s), amplification, transformation or transfection of a host cell, and—if applicable—for integration in a host cell. For example, the vector may comprise regions for integration into host DNA. Integration may be into nuclear DNA of a host cell. In some embodiments, integration may be into non-nuclear DNA, such as chloroplast DNA. Other functional entities of the vectors may include, but are not limited to, markers, linkers and restriction sites.

A target nucleic acid may include a regulatory nucleic acid. This refers to any sequence or nucleic acid which modulates (either directly or indirectly, and either up or down) the replication, transcription and/or expression of a nucleic acid controlled thereby. Control by such regulatory nucleic acid may make a nucleic acid constitutively or inducibly transcribed and/or translated. Any of the nucleotide sequences herein may further comprise a regulatory control sequence. Examples of regulatory control sequences can include, without limitation, one or more of the following: a promoter, an intron, an exon, processing elements, 3′ untranslated region, 5′ untranslated region, RNA stability elements, or translational enhancers. A promoter may be one or more of the following: a promoter adapted for expression in the organism (e.g., bacterial, fungal, viral, plant, mammalian, or protist), an algal promoter, a chloroplast (or other plastid) promoter, a mitochondrial promoter, and a nuclear promoter, any of which may be a native or synthetic promoters. A regulatory control sequence can be inducible or autoregulatable. A regulatory control sequence can include autologous and/or heterologous sequences. In some cases, control sequences can be flanked by a first homologous sequence and a second homologous sequence. The first and second homologous sequences can each be at least 500 nucleotides in length. The homologous sequences can allow for either homologous recombination or can act to insulate the heterologous sequence to facilitate gene expression.

In some instances, target DNA, vector DNA or other DNA present in a shuttle vector of the present disclosure does not result in production of a polypeptide product but rather allows for secretion of the product from the cell. In these cases, the nucleotide sequence may encode a protein that enhances or initiates or increases the rate of secretion of a product from an organism to the external environment.

One embodiment provides a method of producing a gap-filled vector. A gap-filled vector may undergo homologous recombination and insertion of a target nucleic acid according to the invention by filling in the region (gap) between the sequences homologous to the 5′ and the 3′ regions of the target nucleic acid. Hence, in some embodiments, one would contact the instant cloning system with a target nucleic acid under conditions that allow homologous recombination.

Another embodiment combines: (i) a first segment including a first nucleic acid homologous to the 5′ terminus of a target nucleic acid, a first selectable marker and a first cyclization element; (ii) a target nucleic acid; and (iii) a second segment including a second nucleic acid homologous to the 3′ terminus of a target nucleic acid, a second selectable marker and a second cyclization element, under conditions which allow homologous recombination. One embodiment produces a gap-filled vector by homologous recombination between the two arms and the target nucleic acid. The exchange between the homologous regions found in the arms and the target nucleic acid is effected by homologous recombination at any point between the homologous nucleic acids. With respect to a circular vector of the present invention, the “gap filling” essentially is insertion (i.e., subcloning) of the target sequence into the vector.

Homologous recombination may be effected in vitro according to methodologies well known in the art. For example, the methods described herein can be practiced using yeast lysate preparations. Homologous recombination may take place in vivo. Hence, the method of the present disclosure may be practiced using any host cell capable of supporting homologous recombination events such as, for example, bacteria, yeast, plant and mammalian cells. One skilled in the art will appreciate that the choice of a suitable host depends on the particular combination of selectable markers used in the cloning system of the method.

One methodology makes use of a “gene gun” approach. The gene gun is part of a method called the biolistic (also known as bioballistic) method, and under certain conditions, DNA (or RNA) become “sticky,” adhering to biologically inert particles such as metal atoms (usually tungsten or gold). By accelerating this DNA-particle complex in a partial vacuum and placing the target tissue within the acceleration path, DNA is effectively introduced. Uncoated metal particles could also be shot through a solution containing DNA surrounding the cell thus picking up the genetic material and proceeding into the living cell. A perforated plate stops the shell cartridge but allows the slivers of metal to pass through and into the living cells on the other side. The cells that take up the desired DNA, identified through the use of a marker gene (in plants the use of GUS is most common), are then cultured to replicate the gene and possibly cloned. Different methods have been used to accelerate the particles: these include pneumatic devices; instruments utilizing a mechanical impulse or macroprojectile; centripetal, magnetic or electrostatic forces; spray or vaccination guns; and apparatus based on acceleration by shock wave, such as electric discharge (for example, see Christou and McCabe, 1992, Agracetus, Inc. Particle Gun Transformation of Crop Plants Using Electric Discharge (ACCELL™ Technology)).

Transformation can be performed, for example, according to the method of Cohen et al. (Proc. Natl. Acad. Sci. USA, 69:2110 (1972)), the protoplast method (Mol. Gen. Genet., 168:111 (1979)), or the competent method (J. Mol. Biol., 56:209 (1971)) when the hosts are bacteria (E. coli, Bacillus subtilis, and such), the method of Hinnen et al. (Proc. Natl. Acad. Sci. USA, 75:1927 (1978)), or the lithium method (J. Bacteriol., 153:163 (1983)) when the host is S. cerevisiae. Typically, following a transformation event, potential transformants are plated on nutrient media for selection and/or cultivation.

The nutrient media preferably comprises a carbon source, an inorganic nitrogen source, or an organic nitrogen source necessary for the growth of host cells (transformants). Examples of the carbon source are glucose, dextran, soluble starch, and sucrose, and examples of the inorganic or organic nitrogen source are ammonium salts, nitrates, amino acids, corn steep liquor, peptone, casein, meat extract, soy bean cake, and potato extract. If desired, the media may comprise other nutrients (for example, an inorganic salt (for example, calcium chloride, sodium dihydrogenphosphate, and magnesium chloride), vitamins, antibiotics (for example, tetracycline, neomycin, ampicillin, kanamycin, etc.). Media for some photosynthetic organisms may not require a carbon source as such organisms may be photoautotrophs and, thus, can produce their own carbon sources.

Cultivation and/or selection are performed by methods known in the art. Cultivation and selection conditions such as temperature, pH of the media, and cultivation time are selected appropriately for the vectors, host cells and methods of the present invention. One of skill in the art will recognize that there are numerous specific media and cultivation/selection conditions which can be used depending on the type of host cell (transformant) and the nature of the vector (e.g., which selectable markers are present). The media herein are merely described by way of example and are not limiting.

When the hosts are bacteria, actinomycetes, yeast, or filamentous fungi, media comprising the nutrient source(s) mentioned above are appropriate. When the host is E. coli, examples of preferable media are LB media, M9 media (Miller et al. Exp. Mol. Genet., Cold Spring Harbor Laboratory, p. 431 (1972)), and so on. When the host is yeast, an example of medium is Burkhoter minimal medium (Bostian, Proc. Natl. Acad. Sci. USA, 77:4505 (1980)).

The selection of vectors in yeast may be accomplished by the use of yeast selectable markers. Examples include, but are not limited to, TRP1, MET2, MAZF, ADE2, ADE6, URA3, URA3, ARG1, ARG2, ARG3, HIS1, HIS2, HIS3, HIS5, HIS6, and LEU2. In certain embodiments, the HIS3, TRP1, URA3, LEU2 and ADE2 markers are used. In some embodiments, a vector or segment thereof may comprise two or more selectable markers. Thus, in one embodiment, a segment of a vector of the present invention may comprise an ADE marker to be lost upon homologous recombination with the target nucleic acid, and a HIS3 marker. The other segment may comprise a TRP1 marker. Selection is achieved by growing transformed cells on a suitable drop-out selection media (see e.g., Watson et al. (1992) Recombinant DNA, 2.sup.nd ed., Freeman and Co., New York, N.Y.). For example, HIS3 allows for selection of cells containing the first segment. TRP1 allows for selection of cells containing the second segment. ADE allows screening and selection of clones in which homologous recombination took place. ADE enables color selection (red). In some embodiments, pairs of selection markers comprising the URA3 gene in combination with a second marker are utilized. URA3 is used in each pair because it allows for both positive and negative selection. The URA3 gene is then coupled with a second marker such as the LEU2, HIS3, LYS2 or kanMX6 marker. Either member of the pair can be used for selection when a single modification is introduced or just the non-URA3 marker in the case of two or more modifications.

Recombinant yeast cells may be selected using the selectable markers described herein according to methods well known in the art. Hence, one skilled in the art will appreciate that recombinant yeast cells harboring a gap-filled vector of the invention may be selected on the basis of the selectable markers included therein. For example, recombinant vectors carrying HIS3 and TRP1 may be selected by growing transformed yeast cells in the presence of drop-out selection media lacking histidine and tryptophan. Isolated positive clones may be purified further and analyzed to ascertain the presence and structure of the recombinant vector of the invention by, e.g., restriction analysis, electrophoresis, Southern blot analysis, polymerase chain reaction or the like. Also provided are gap-filled vectors engineered according to the methods described herein. Such a vector is the product of homologous recombination between the segments or vectors of the present disclosure and a target nucleic acid of choice. Also provided is a prokaryotic cell and/or a eukaryotic host cell harboring the cloning system or vector according to the present disclosure. The organism can be unicellular or multicellular. The organism may be naturally photosynthetic or naturally non-photosynthetic. Other examples of organisms that can be transformed include vascular and non-vascular organisms. When hosts, such as plant, yeast, or algal cells are used, a vector may contain, at least, a promoter, an initiation codon, the polynucleotide encoding a protein, and a termination codon. The vectors may also contain, if required, a polynucleotide for gene amplification (marker) that is usually used.

### Products

The vectors described herein may comprise sequences that result in production of a product naturally, or not naturally, produced in the organism comprising the vector. In some instances the product encoded by one or more sequences on a vector is a polypeptide, for example an enzyme. Enzymes utilized may be encoded by nucleotide sequences derived from any organism, including bacteria, plants, fungi and animals. Vectors may also comprise nucleotide sequences that affect the production or secretion of a product from the organism. In some instances, such nucleotide sequence(s) encode one or more enzymes that function in isoprenoid biosynthetic pathway. Examples of polypeptides in the isoprenoid biosynthetic pathway include synthases such as C5, C10, C15, C20, C30, and C40 synthases. In some instances, the enzymes are isoprenoid producing enzymes. In some instances, an isoprenoid producing enzyme produces isoprenoids with two phosphate groups (e.g., GPP synthase, FPP synthase, DMAPP synthase). In other instances, isoprenoid producing enzymes produce isoprenoids with zero, one, three or more phosphates or may produce isoprenoids with other functional groups. Polynucleotides encoding enzymes and other proteins useful in the present invention may be isolated and/or synthesized by any means known in the art, including, but not limited to cloning, sub-cloning, and PCR.

An isoprenoid producing enzyme may also be botryococcene synthase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, γ-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elisabethatriene synthase, sandalol synthase, patchoulol synthase, zinzanol synthase, cedrol synthase, scareol synthase, copalol synthase, or manool synthase.

Other enzymes which may be produced by vectors of the present disclosure include biomass-degrading enzymes. Non-limiting examples of biomass-degrading enzymes include: cellulolytic enzymes, hemicellulolytic enzymes, pectinolytic enzymes, xylanases, ligninolytic enzymes, cellulases, cellobiases, softening enzymes (e.g., endopolygalacturonase), amylases, lipases, proteases, RNAses, DNAses, inulinase, lysing enzymes, phospholipases, pectinase, pullulanase, glucose isomerase, endoxylanase, beta-xylosidase, alpha-L-arabinofuranosidase, alpha-glucoronidase, alpha-galactosidase, acetylxylan esterase, and feruloyl esterase. Examples of genes that encode such enzymes include, but are not limited to, amylases, cellulases, hemicellulases, (e.g., β-glucosidase, endocellulase, exocellulase), exo-β-glucanase, endo-β-glucanase and xylanse (endoxylanase and exoxylanse). Examples of ligninolytic enzymes include, but are not limited to, lignin peroxidase and manganese peroxidase from Phanerochaete chryososporium. One of skill in the art will recognize that these enzymes are only a partial list of enzymes which could be used.

The present disclosure contemplates making enzymes that contribute to the production of fatty acids, lipids or oils by transforming host cells and/or organisms comprising host cells with nucleic acids encoding one or more different enzymes. In some embodiments the enzymes that contribute to the production of fatty acids, lipids or oils are anabolic enzymes. Some examples of anabolic enzymes that contribute to the synthesis of fatty acids include, but are not limited to, acetyl-CoA carboxylase, ketoreductase, thioesterase, malonyltransferase, dehydratase, acyl-CoA ligase, ketoacylsynthase, enoylreductase and a desaturase. In some embodiments the enzymes are catabolic or biodegrading enzymes. In some embodiments, a single enzyme is produced.

Some host cells may be transformed with multiple genes encoding one or more enzymes. For example, a single transformed cell may contain exogenous nucleic acids encoding enzymes that make up an entire fatty acid synthesis pathway. One example of a pathway might include genes encoding an acetyl CoA carboxylase, a malonyltransferase, a ketoacylsynthase, and a thioesterase. Cells transformed with entire pathways and/or enzymes extracted from them, can synthesize complete fatty acids or intermediates of the fatty acid synthesis pathway. In some embodiments constructs may contain multiple copies of the same gene, and/or multiple genes encoding the same enzyme from different organisms, and/or multiple genes with mutations in one or more parts of the coding sequences.

In some instances, a product (e.g. fuel, fragrance, insecticide) is a hydrocarbon-rich molecule, e.g. a terpene. A terpene (classified by the number of isoprene units) can be a hemiterpene, monoterpene, sesquiterpene, diterpene, triterpene, or tetraterpene. In specific embodiments the terpene is a terpenoid (aka isoprenoid), such as a steroid or carotenoid. Subclasses of carotenoids include carotenes and xanthophylls. In specific embodiments, a fuel product is limonene, 1,8-cineole, α-pinene, camphene, (+)-sabinene, myrcene, abietadiene, taxadiene, farnesyl pyrophosphate, amorphadiene, (E)-α-bisabolene, beta carotene, alpha carotene, lycopene, or diapophytoene. Some of these terpenes are pure hydrocarbons (e.g. limonene) and others are hydrocarbon derivatives (e.g. cineole).

Examples of fuel products include petrochemical products and their precursors and all other substances that may be useful in the petrochemical industry. Fuel products include, for example, petroleum products, and precursors of petroleum, as well as petrochemicals and precursors thereof. The fuel product may be used for generating substances, or materials, useful in the petrochemical industry, including petroleum products and petrochemicals. The fuel or fuel products may be used in a combustor such as a boiler, kiln, dryer or furnace. Other examples of combustors are internal combustion engines such as vehicle engines or generators, including gasoline engines, diesel engines, jet engines, and others. Fuel products may also be used to produce plastics, resins, fibers, elastomers, lubricants, and gels.

Examples of products contemplated herein include hydrocarbon products and hydrocarbon derivative products. A hydrocarbon product is one that consists of only hydrogen molecules and carbon molecules. A hydrocarbon derivative product is a hydrocarbon product with one or more heteroatoms, wherein the heteroatom is any atom that is not hydrogen or carbon. Examples of heteroatoms include, but not limited to, nitrogen, oxygen, sulfur, and phosphorus. Some products are hydrocarbon-rich, wherein as least 50%, 60%, 70%, 80%, 90%, or 95% of the product by weight is made up carbon and hydrogen.

Fuel products, such as hydrocarbons, may be precursors or products conventionally derived from crude oil, or petroleum, such as, but not limited to, liquid petroleum gas, naptha (ligroin), gasoline, kerosene, diesel, lubricating oil, heavy gas, coke, asphalt, tar, and waxes. For example, fuel products may include small alkanes (for example, 5 to approximately 4 carbons) such as methane, ethane, propane, or butane, which may be used for heating (such as in cooking) or making plastics. Fuel products may also include molecules with a carbon backbone of approximately 5 to approximately 9 carbon atoms, such as naptha or ligroin, or their precursors. Other fuel products may be about 5 to about 12 carbon atoms or cycloalkanes used as gasoline or motor fuel. Molecules and aromatics of approximately 10 to approximately 18 carbons, such as kerosene, or its precursors, may also be fuel products. Fuel products may also include molecules, or their precursors, with more than 12 carbons, such as used for lubricating oil. Other fuel products include heavy gas or fuel oil, or their precursors, typically containing alkanes, cycloalkanes, and aromatics of approximately 20 to approximately 70 carbons. Fuel products also includes other residuals from crude oil, such as coke, asphalt, tar, and waxes, generally containing multiple rings with about 70 or more carbons, and their precursors.

In one embodiment, the vector comprises a nucleotide sequence encoding an enzyme utilized in the production of plant sterols or phytosterols. As used herein the term “phytosterols” includes in addition to phytosterols, phytosterol esters, phytostanols and phytostanol esters. Phytosterols have been shown in clinical trials to reduce absorption of cholesterol and are approved by the U.S. Food and Drug administration for use as a food additive. In other embodiments, the vectors comprises nucleotide sequences encoding therapeutic proteins. Numerous therapeutic proteins are known in the art and include, but are not limited to, hormones such as insulin and erythropoietin, antibodies, vaccines, albumins and interferons.

Host Cells and Organisms

Examples of organisms that can be transformed using the vectors and methods herein include vascular and non-vascular organisms. The organism can be prokaryotic or eukaryotic. The organism can be unicellular or multicellular.

Eukaryotic cells, such as a fungal cell (e.g., Saccharomyces cerevisiae, Schizosaccharomyces pombe or Ustilago maydis) may be transformed using the methods and compositions disclosed herein. Methods for introducing nucleic acids in a fungal/yeast cells are well known in the art. Hence, such a step may be accomplished by conventional transformation methodologies. Non-limiting examples of suitable methodologies include electroporation, alkali cations protocols and spheroplast transformation.

Examples of non-vascular photosynthetic organisms include bryophytes, such as marchantiophytes or anthocerotophytes. In some instances the organism is a cyanobacteria. In some instances, the organism is algae (e.g., macroalgae or microalgae). The algae can be unicellular or multicellular algae. In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

The use of microalgae to express a polypeptide or protein complex according to a method disclosed herein provides the advantage that large populations of the microalgae can be grown, including commercially (Cyanotech Corp.; Kailua-Kona Hi.), thus allowing for production and, if desired, isolation of large amounts of a desired product. However, the ability to express, for example, functional polypeptides, including protein complexes, in the chloroplasts of any plant and/or modify the chloroplasts or any plant allows for production of crops of such plants and, therefore, the ability to conveniently produce large amounts of the polypeptides. Accordingly, the methods of the invention can be practiced using any plant having chloroplasts, including, for example, macroalgae, for example, marine algae and seaweeds, as well as plants that grow in soil, for example, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos mucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugar cane (Saccharum spp.), oats, duckweed (Lemna), barley, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals such as azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum are also included. Additional ornamentals useful for practicing a method of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Leguminous plants that may be used include beans and peas. Beans include guar, locust bean, fenugreek, soybean, garden beans, cowpea, mung bean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Exemplary forage and turf grass include alfalfa, orchard grass, tall fescue, perennial ryegrass, creeping bent grass, and redtop. Other plants useful in the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, chicory, groundnut and zucchini. Thus, the compositions contemplated herein include host organisms comprising any of the above vectors. The host organism can be any chloroplast-containing organism.

The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

Eukaryotic host cells may be a fungal cell (e.g., S. cerevisiae, Sz. pombe or U. maydis). Examples of prokaryotic host cells include E. coli and B. subtilis, cyanobacteria and photosynthetic bacteria (e.g. species of the genus Synechocystis or the genus Synechococcus or the genus Athrospira). Examples of non-vascular plants which may be a host organism (or the source of target DNA) include bryophytes, such as marchantiophytes or anthocerotophytes. In some instances, the organism is algae (e.g., macroalgae or microalgae, such as Chlamydomonas reinhardtii, Chorella vulgaris, Dunaliella salina, Haematococcus pluvalis, Scenedesmus ssp.). The algae can be unicellular or multicellular algae. In some instances the organism is a rhodophyte, chlorophyte, heterokontophyte, tribophyte, glaucophyte, chlorarachniophyte, euglenoid, haptophyte, cryptomonad, dinoflagellum, or phytoplankton. In other instances one of skill in the art will recognize that these organisms are given merely as examples and other organisms may be substituted where appropriate positive and negative selectable markers are available.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLES

### Example 1

### DNA Purification and Analysis

DNA is isolated and analyzed according to methods known in the art. Various methods for the isolation of nuclear and plastid DNA from plants have been published and are available to the skilled artisan for in example in Murray and Thompson, Nuc. Acids Res., 8:4321, 1980; Bovenberg et al., Nuc. Acids Res. 9:503, 1981; Triboush et al., Plant Molec Biol. Reporter, 16:183, 1998; and Methods in Plant Molecular Biology, A Laboratory Course Manual, Maliga et al, Cold Spring Harbor Laboratory Press, 1995.

To prepare DNA from plant cells, the DNeasy Plant Mini Kit is used (Qiagen).

To prepare DNA from yeast to use as a template for PCR, 10⁶ yeast cells (from agar plate or liquid culture) are suspended in lysis buffer (6 mM KHPO4, pH=7.5, 6 mM NaCl, 3% glycerol, 1 U/mL zymolyase) and heated to 37° C. for 30 ml, 95° C. for 10 minutes, then cooled to near 23° C. The solution is added to the PCR mixture directly.

To prepare plasmid DNA from yeast, desired clones are grown in selective liquid media (e.g., CSM-Trp) to saturation at 30° C. Cells are collected by centrifugation at 3000×g for 10 minutes and resuspended in 150 uL of lysis buffer (1 M sorbitol, 0.1 M sodium citrate, 0.06 M EDTA pH=7.0, 100 mM beta-mercaptoethanol, and 2.5 mg/mL zymolyase). The solution is incubated for 1 hr at 37° C. 300 uL of denaturing solution (1% SDS and 0.2N NaOH) is added and solution is incubated at 60° C. for 15 min. 150 uL of neutralizing solution (3M potassium acetate, pH=4.8) is added and the solution is incubated on ice for 10 min. The solution is centrifuged at 14,000 RPM for 10 min and the supernatant is transferred to another tube. 1 mL of isopropanol is added, the mixture is gently mixed and centrifuged at 14,000 RPM for 10 min. The pellet is washed once with 1 mL of 70% ethanol and centrifuged at 14,000 RPM for 10 min. The DNA pellet is air-dried and resuspended in 60 uL of resuspension buffer (10 mM Tris pH=7.4, 1 mM EDTA, and 0.1 mg/mL RNase).

To prepare plasmid DNA from bacteria, cells are grown to saturation at 37° C. in LB containing the appropriate antibiotic (Kan or Amp). If the DNA of interest contains standard replication elements, cells are harvested by centrifugation. If the DNA of interest contains P1 replication elements, saturated cell cultures are diluted 1:20 in LB+Kan+IPTG and grown for 4 hours at 37° C., then harvested. The Plasmid Maxi kit (QIAGEN) is used to prepare plasmid DNA from the cell pellets.

For illustrative purposes, and without limiting the invention to the specific methods described, DNA samples prepared from algae, yeast, or bacteria (in plugs or in solution) are analyzed by pulse-field gel electrophoresis (PFGE), or digested with the appropriate restriction endonuclease (e.g., SmaI) and analyzed by PFGE, conventional agarose gel electrophoresis, and/or Southern blot. Standard protocols useful for these purposes are fully described in Gemmill et al. (in “Advances in Genome Biology”, Vol. 1, “Unfolding The Genome,” pp 217 251, edited by Ram S. Verma).

One of skill will appreciate that many other methods known in the art may be substituted in lieu of the ones specifically described or referenced.

### Example 2

### Transformation Methods

E. coli strains DH 10B or Genehog are made electrocompetent by growing the cells to an OD₆₀₀ of 0.7, then collected and washed twice with ice-cold 10% glycerol, flash frozen in a dry-ice ethanol bath and kept at −80° C. Total yeast or algae DNA is prepared and electroporated into E. coli by using, for example, a 0.1 cm cuvette at 1,800 V, 200 ohms and 25 mF in a Bio-Rad Gene Pulsar Electroporator. Cells are allowed to recover and clones are selected on agar growth media containing one or more antibiotics, such as kanamycin (50 μg/mL), ampicillin (100 μg/mL), gentamycin (50 μg/mL), tetracycline (51 μg/mL), or chloramphenicol (34 μg/mL).

Yeast strains YPH857, YPH858 or AB1380 may be transformed by the lithium acetate method as described in Sheistl & Geitz (Curr. Genet. 16:339 346, 1989) and Sherman et al., “Laboratory Course Manual Methods in Yeast Genetics” (Cold Spring Harbor Laboratory Press, Cold Spring Harbor, 1986) or a spheroplast method such as the one described by Sipiczki et al., Curr. Microbiol., 12(3):169-173 (1985). Yeast transformants are selected and screened on agar media lacking amino and/or nucleic acids, such as tryptophan, leucine, or uracil. Standard methods for yeast growth and phenotype testing are employed as described by Sherman et al., supra.

Chloroplasts of plants are transformed based on the method of Maenpaa et al., Molec. Biotech., 13:67, 1999. Tungsten or gold particles are sterilized for use as microcarriers in bombardment experiments. Particles (50 mg) are sterilized with 1 ml of 100% ethanol, and stored at −20° C. or −80° C. Immediately prior to use, particles are sedimented by centrifugation, washed with 2 to 3 washes of 1 ml sterile deionised distilled water, vortexed and centrifuged between each wash. Washed particles are resuspended in 500 ul 50% glycerol.

Sterilized particles are coated with DNA for transformation. 25 ul aliquots of sterilized particles are added to a 1.5 ml microfuge tube, and 5 ug of DNA of interest is added and mixed by tapping. 35 ul of a freshly prepared solution of 1.8M CaCl₂ and 30 mM spermidine is added to the particle/DNA mixture, mixed gently, and incubated at room temperature for 20 minutes. The coated particles are sedimented by centrifuging briefly. The particles are washed twice by adding 200 ul 70% ethanol, mixing gently, and centrifuging briefly. The coated particles are resuspended in 50 ul of 100% ethanol and mixed gently. Five to ten microliters of coated particles are used for each bombardment.

Transformation by particle bombardment is carried out using the PDS 1000 Helium gun (Bio Rad, Richmond, Calif.) using a modified protocol described by the manufacturer. Plates containing leaf samples are placed on the second shelf from the bottom of the vacuum chamber and bombarded using the 1100 p.s.i. rupture disk. After bombardment, petriplates containing the leaf samples are wrapped in plastic bags and incubated at 24° C. for 48 hours.

After incubation, bombarded leaves are cut into approximately 0.5 cm² pieces and placed abaxial side up on selection medium. After 3 to 4 weeks on the selection medium, small, green shoots will appear on the leaf tissue. These shoots will continue to grow on selection medium and are referred to as putative transformants. When the putative transformants have developed 2 to 3 leaves, 2 small pieces (approximately 0.5 cm²) are cut from each leaf and used for additional rounds of shoot regeneration or further growth.

Two to four shoots of each positive transformant are selected and transferred to selection medium for generation of roots. Analysis is performed on 2 shoots to confirm homoplasmy. Shoots from homoplasmic events are transferred to the greenhouse for seed production, while transformants which are not homoplasmic are sent through a second round or regeneration on selection medium to attain homoplasmy.

One of skill will appreciate that many other transformation methods known in the art may be substituted in lieu of the ones specifically described or referenced herein.

### Example 3

### Hybrid Vector System

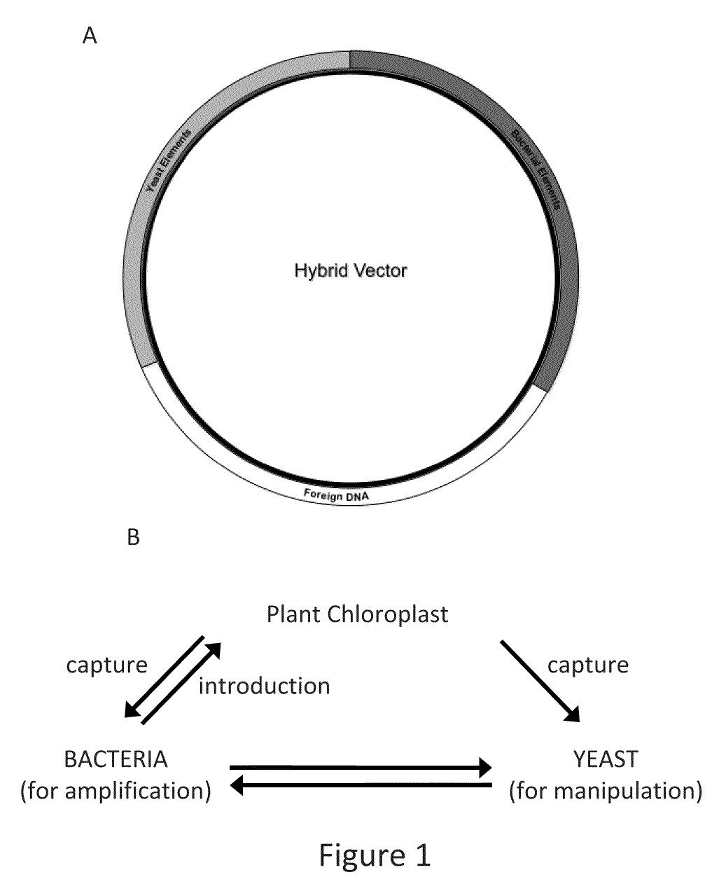

In this example, a system is established using a hybrid vector to support replication of chloroplast DNA from vascular plants in yeast and bacteria (FIG. 1). The hybrid gap filling vector backbone contains yeast elements that allow it to function as a yeast artificial plasmid (YAP) and bacterial elements that allow it to function as a plasmid artificial chromosome (PAC) or a bacterial artificial chromosome (BAC). The yeast elements include a yeast selection marker sequence (e.g. TRP1 or LEU2), a yeast centromere sequence (CEN), and a yeast autonomously replicating nucleotide sequence (ARS). Bacterial elements include the P1 origin of replication or the F′ bacterial origin of replication sequences and a bacterial selection maker sequence (e.g. Cam^{r} or Kan^{r}). The yeast and bacterial elements may be combined with chloroplast DNA in a single vector by a variety of methods, including, but not limited to, integration of yeast elements into existing BAC and/or PAC clones containing chloroplast DNA (described below), capture of chloroplast DNA fragments into a hybrid vector by homologous recombination in yeast (aka gap-filling), and constructing of large DNA fragment libraries using a vector containing yeast and bacterial elements as the backbone.

To obtain chloroplast DNA in a hybrid vector by integrating yeast elements into existing BAC clones, homologous recombination in yeast may be used. The region of DNA in pTrp-10-Kan (SEQ ID NO. 1) that contains sequences encoding ARS, CEN and TRP1 is PCR amplified using primers (SEQ ID NOs. 2 and 3) with 5′ tails homologous to sequences within pBeloBAC-11 (SEQ ID NO. 4) or its derivatives. The PCR product is then mixed with the desired BAC clone and transformed into yeast. Transformants are identified by growth on selective media. Once confirmed, DNA from desired yeast clones is prepared and transformed into bacteria for amplification and purification.

To increase recombination efficiency and obtain modified BACs more efficiently, longer regions of homology were used. A 5.0 kb region that contains the yeast replication and selection elements and terminal sequences homologous pBeloBAC-11 was PCR amplified from a BAC modified by the PCR method described above (previous paragraph) using primers (SEQ ID NOs. 5 and 6) that both have SnaBI and XhoI sites at the 5′ ends. The PCR product was purified by ethanol precipitation, digested with SnaBI, and isolated using agarose gel electrophoresis and the QIAQUICK gel extraction kit (QIAGEN). The SnaBI digested DNA fragment was then ligated to pUC-SE (SEQ ID NO. 7) that was digested with NotI and treated with Klenow enzyme to create blunt ends, creating pBeloYAP (SEQ ID NO. 8). The integration cassette is liberated from pBeloYAP by digestion with XhoI and isolated using agarose gel electrophoresis and the QIAQUICK gel extraction kit (QIAGEN). The 5.0 kb restriction fragment is mixed with the desired BAC clone and transformed into yeast. Transformants are identified by growth on selective media. Once confirmed, DNA from desired yeast clones is prepared and transformed into bacteria for amplification and purification.

One of skill will appreciate that many other methods known in the art may be substituted in lieu of the ones specifically described or referenced.

### Example 4

### Vectors to Stabilize and/or Modify Chloroplast Genome DNA in an Exogenous Host

Often, large pieces of heterologous DNA are instable in host organisms such as yeast or bacteria. This may be due to multiple factors, including, but not limited to, the presence of toxic gene products or codon bias and/or lack of selective pressure. Therefore, the target DNA within the shuttle vector may be altered within yeast or bacteria. For example, certain portions of a target DNA sequence (e.g., coding regions or promoters) may be deleted or moved by recombination within the host organism. In a similar way, when a shuttle vector carrying the target DNA is transferred back to the organism (or a closely related species) that donated the target DNA, the target DNA can become unstable.

Pairs of yeast selection markers were constructed so that multiple stabilization sites could be employed simultaneously. Each marker pair contains the URA3 gene (SEQ ID NO. 9), which was PCR amplified from pRS416. Each marker pair also contains the LEU2 gene (SEQ ID NO. 10) amplified from pRS415, the HIS3 gene (SEQ ID NO. 11) amplified from pRS413, the ADE2 gene (SEQ ID NO. 12) amplified from pTrp-AU, the LYS2 gene (SEQ ID NO. 13) amplified from S. cerevisiae genomic DNA, or the kanMX6 gene (SEQ ID NO. 14) from pFA6a-kanMX6, which confers resistance to the antifungal agent G418. The primers used for the URA3 gene add the XmaI restriction site to the 5′ end (SEQ ID NO. 15) and SalI and SacII to the 3′ end (SEQ ID NO. 16). The primers used for the LEU2, HIS3, ADE2, LYS2, and G418^{r} genes add the XmaI restriction site to the 5′ end (SEQ ID NO. 17 for LEU2, SEQ ID NO. 18 for HIS3, SEQ ID NO. 19 for ADE2, SEQ ID NO. 20 for LYS2, and SEQ ID NO. 21 for G418^{r}) and SalI, FseI, and SpeI sites to the 3′ end (SEQ ID NO. 22 for LEU2, SEQ ID NO. 23 for HIS3, SEQ ID NO. 24 for ADE2, SEQ ID NO. 25 for LYS2, and SEQ ID NO. 26 for G418^{r}). Each PCR product was digested with XmaI and SalI, mixed pairwise, and ligated into SalI-digested pUC1 (SEQ ID NO. 27). URA3 is used in each case because it allows for positive and negative selection. Marker pairs can be introduced based on selection for either gene (in the case of a single modification), the non-URA3 gene in the case of two or more modification. Then the markers can be removed by introducing DNA with terminal sequences homologous to those surrounding the marker pairs and selecting for growth on minimal media containing 5-FOA.

To promote sequence stability in bacteria, antibiotic resistance markers were cloned into the yeast selection marker pairs. The bacterial stability markers include, but are not limited to, the ampicillin resistance gene (Amp^{r}, SEQ ID NO. 14) amplified from pET-21a, the tetracycline resistance gene (Tet^{r}, SEQ ID NO. 27) amplified from pBR322, the chloramphenicol resistance gene (Cam^{r}, SEQ ID NO. 28) amplified from pETcoco-1, and the gentamycin resistance gene (Gent^{r}, SEQ ID NO. 29) amplified from pJQ200. For each gene, primer pairs (SEQ ID NOs. 30 and 31 for Amp^{r}, SEQ ID NOs. 32 and 33 for Tet^{r}, SEQ ID NOs. 34 and 35 for Cam^{r}, and SEQ ID NOs. 36 and 37 for Gent^{r}) that add XmaI sites to both the 5′ and 3′ ends were used to PCR amplify the antibiotic resistance fragment. Each PCR product was digested with XmaI and ligated into XmaI-digested vectors containing yeast marker pair cassettes.

The yeast selection marker cassettes (with or without the bacterial antibiotic resistance markers) may be cloned into traditional targeting vectors for integration into the desired target sequence by homologous recombination in yeast. In addition, these same selection marker cassettes may serve as the template for a one-step PCR-mediated technique for introducing selection markers into target regions. Two PCR primers are designed such that the sequence of the first 40-42 nucleotides (5′->3′) of each primer are identical to the target sequences, and the final 18-20 nucleotides are identical to sequences within a vector containing a selection marker cassette. Thus, PCR amplification of the selection marker cassette adds flanking sequences that target the selection marker(s) to the desired region.

### Example 5

### Cloning the Glycine Max Chloroplast Genome with a Hybrid Cloning System

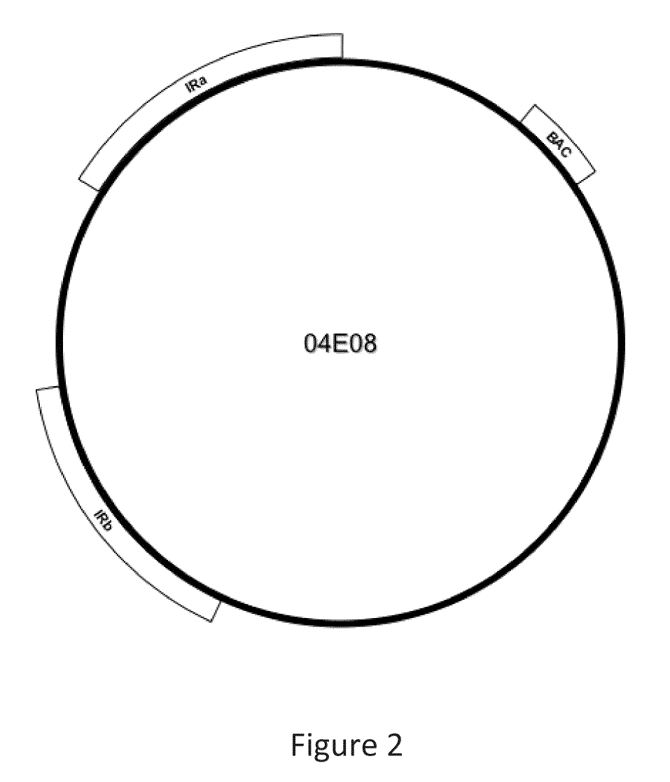

In this example, a system is established using a hybrid vector to support replication of chloroplast DNA from G. max in yeast and bacteria (FIG. 1). G. max chloroplast genome DNA was derived from BAC clone 04E08 from a BAC library derived from G. max total genomic DNA (described in Saski et al., Plant Mol. Bio., 59: 309-322 (2005)) (FIG. 2). Clone 04E08 contains the entire chloroplast genome in a single, circular molecule that stably replicates in bacteria. Incorporation of yeast elements for replication and selection was thus required to adapt clone 04E08 to the hybrid system.

To incorporate the required yeast elements, we used homologous recombination in yeast. First, pBeloYAP was digested with XhoI, liberating a 5.0 kb fragment containing a yeast ARS and CEN, TRP1 selection marker, and flanking sequences homologous to those found in the pIndigoBAC vector backbone. This fragment was isolated using agarose gel electrophoresis and the QIAQUICK gel extraction kit (QIAGEN). Next, the URA3-LEU2 selection marker cassette was PCR amplified using primers (SEQ ID NOs. 38 and 39) with 5′ tails homologous to sequences flanking G. max chloroplast genome nucleotide 122,283 (according to the sequence available from NCBI, NC_{—}007942). The PCR product was isolated using agarose gel electrophoresis and the QIAQUICK gel extraction kit (QIAGEN).

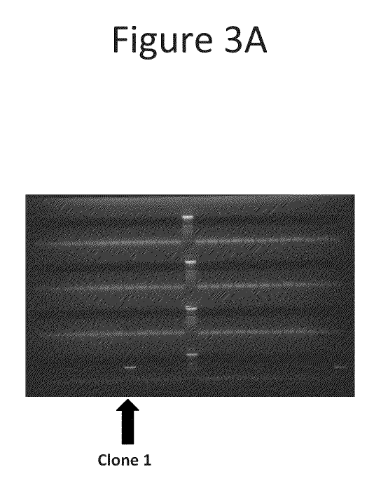

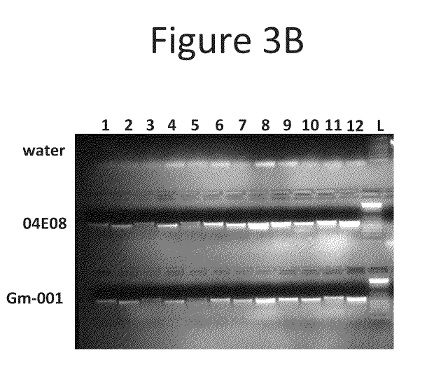

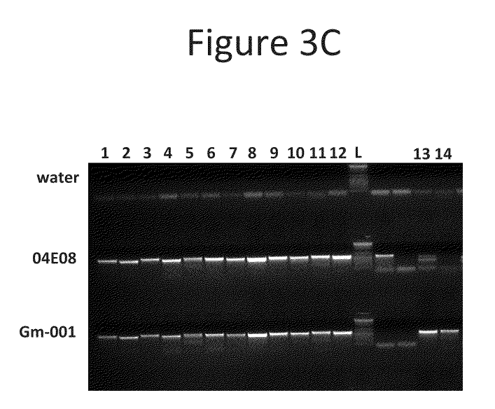

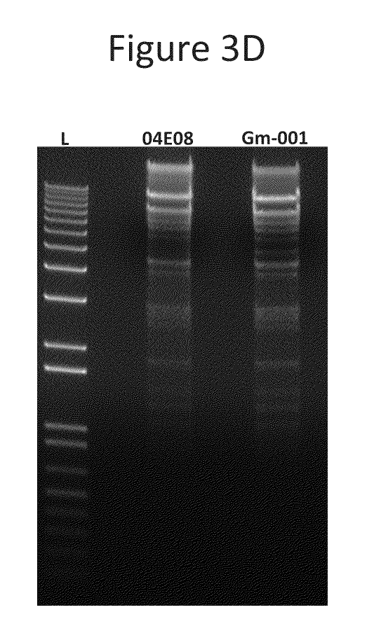

The DNA fragments containing the yeast replication and selection elements were mixed with clone 04E08 and transformed into YPH858 using the spheroplast method. Transformants were identified by growth on CSM-ura-leu agar medium and propagated on CSM-ura-leu-trp agar medium. Yeast clones that grow on both media types were screened by colony PCR using primers (SEQ ID NOs. 40 and 41) that amplify a region within the G. max chloroplast genome surrounding nucleotide 000,060 (according to the sequence available from NCBI, NC_{—}007942). Desired clones are those that give rise to a PCR product of expected size. FIG. 3A shows that clone 1 gave rise to a PCR product. DNA was prepared from Clone 1 grown in CSM-ura-leu-trp liquid medium and screened with additional primers (SEQ ID NOs. 40 and 41; 42 and 43; 44 and 45; 46 and 47; 48 and 49; 50 and 51; 52 and 53; 54 and 55; 56 and 57; 58 and 59; 60 and 61; and 62 and 63) spread throughout the G. max chloroplast genome. Desired clones are those that give rise to PCR products of expected size for all reaction. FIG. 3B shows that clone 1 gave rise to a PCR product of expected size in all reactions, indicating that clone 1 harbors clone 04E08 with integrated yeast replication and selection elements, hereafter called Gm-001.

Gm-001 was transferred to bacterial strain DH10B by electroporation, followed by selection on LB agar medium containing chloramphenicol (34 μg/mL). Isolated transformants were screened by colony PCR using primers (SEQ ID NOs. 40 and 41; 42 and 43; 44 and 45; 46 and 47; 48 and 49; 50 and 51; 52 and 53; 54 and 55; 56 and 57; 58 and 59; 60 and 61; and 62 and 63) spread throughout the G. max chloroplast genome and primers (SEQ ID NOs. 64 and 65 and SEQ ID NOs. 66 and 67) specific for integration of the URA3-LEU2 cassette. Desired clones are those that give rise to PCR products of expected size for all reaction. FIG. 3C shows a bacterial clone gave rise to a PCR product of expected size in all reactions, demonstrating that Gm-001 was indeed transferred from yeast into bacteria.

To further confirm that Gm-001 is stable in the hybrid system, the DNA molecule was isolated and analyzed by restriction mapping. Briefly, bacterial clones were growing to saturation in LB media containing chloramphenicol and collected by centrifugation. The Plasmid Maxi kit (QIAGEN) is used to prepare plasmid DNA from the isolated clones. Each molecule, as well as clone 04E08, was restriction mapped with HindIII. FIG. 3D shows that the isolated clone appear similar to clone 04E08, indicating that Gm-001 is stable in the hybrid system.

One of skill will appreciate that many other methods known in the art may be substituted in lieu of the ones specifically described or referenced.

### Example 6

### Cloning the A. thaliana Chloroplast Genome Using a Hybrid System (Prophetic)

In this example, a system is established using a hybrid vector to support replication of chloroplast DNA from A. thaliana in yeast and bacteria. The A. thaliana chloroplast genome DNA is derived from three PAC clones, MAB17, MC13, and MAH2, which are obtained from the Mitsui P1 library (described in Liu et al., Plant J., 7: 351-358 (1995) and Sato et al., DNA Res., 6: 283-290 (1999)). These three PAC clones replicates stably in bacteria and contain partially overlapping sequences that comprise the entire A. thaliana chloroplast genome. MAB17, MC13 and MAH2 are digested with restriction endonucleases to generate linear DNA fragments with overlapping 5′ and 3′ termini. These restriction fragments are transformed into yeast with a hybrid vector backbone containing DNA sequences homologous to adjacent regions in the A. thaliana chloroplast genome. A single DNA molecule comprising the entire A. thaliana chloroplast genome is created by homologous recombination.

Standard methods are used to identify desired clones.

One of skill will appreciate that many other methods known in the art may be substituted in lieu of the ones specifically described or referenced.

### Example 7

### Cloning the Z. mays Chloroplast Genome Using a Hybrid System

In this example, a system is established using a hybrid vector to support replication of chloroplast DNA from Z. mays in yeast and bacteria. A hybrid gap-filling vector is created with DNA sequences that have high homology to adjacent regions in the Z. mays chloroplast genome. Chloroplast genome DNA is obtained from genomic DNA preparations from Z. mays cells. A single DNA molecule comprising the entire Z. mays chloroplast genome is created by transforming linearized gap filling vector DNA and chloroplast genome DNA into S. cerevisiae using the lithium acetate or spheroplast methods as described in EXAMPLE 2. Homologous recombination takes place in vivo in the transformed yeast cells. Once the target DNA is captured by the vector via homologous recombination, the DNA can be stably replicated in both yeast and bacterial systems.

Standard methods are used to identify desired clones.

One of skill will appreciate that many other methods known in the art may be substituted in lieu of the ones specifically described or referenced.

### Example 8

### Cloning the O. sativa Chloroplast Genome Using a Hybrid System

In this example, a system is established using a hybrid vector to support replication of chloroplast DNA from O. sativa in yeast and bacteria. A hybrid gap-filling vector is created with DNA sequences that have high homology to adjacent regions in the O. sativa chloroplast genome. Chloroplast genome DNA is obtained from genomic DNA preparations from O. sativa cells. A single DNA molecule comprising the entire O. sativa chloroplast genome is created by transforming linearized gap filling vector DNA and chloroplast genome DNA into S. cerevisiae using the lithium acetate or spheroplast methods as described in EXAMPLE 2. Homologous recombination takes place in vivo in the transformed yeast cells. Once the target DNA is captured by the vector via homologous recombination, the DNA can be stably replicated in both yeast and bacterial systems.

Standard methods are used to identify desired clones.

One of skill will appreciate that many other methods known in the art may be substituted in lieu of the ones specifically described or referenced.

What is claimed is:

1. A vector comprising all essential genes of a plastid genome from a vascular plant, wherein said vector further comprises at least one yeast selection marker sequence, a yeast centromere sequence, a yeast autonomously replicating nucleotide sequence, a bacterial origin of replication and at least one bacterial selection marker, wherein said vector provides for stable replication of said plastid genome in a yeast and a bacterial cell.

2. The vector of claim 1, wherein said plastid genome is at least 135 kb in size.

3. The vector of claim 1, wherein said plastid genome is at least 150 kb in size.

4. The vector of claim 1, wherein said plastid is a chloroplast.

5. The vector of claim 1 wherein said genome comprises at least about 90% of a chloroplast genome.

6. The vector of claim 1, wherein said yeast selection marker is at least one of the group consisting of HIS3, TRP1, URA3, ADE2 and LEU2.

7. The vector of claim 1, wherein said bacterial selection marker is at least one of the group consisting of tetracycline resistance, ampicillin resistance, chloramphenicol resistance, kanamycin resistance and neomycin resistance.

8. The vector of claim 1, wherein said bacterial origin of replication is a P1 or F′ origin of replication.

9. The vector of claim 1, wherein said plastid genome is from a vascular plant selected from the group consisting of soybeans, tomatoes, potatoes, wheat, rice, corn, barley, oats, rye, cotton, Arabidopsis, tobacco, and legumes

10. The vector of claim 1, wherein said plastid genome is a chloroplast genome.

11. The vector of 10, wherein said chloroplast genome is from a soybean.

12. The vector of claim 1, wherein said vector comprises at least one pair of yeast selection markers.

13. The vector of claim 12, wherein said at least one pair of yeast selection markers allow for positive and negative selection.

14. The vector of claim 13, wherein each yeast selection marker pair comprises a URA3 gene.

15. The vector of claim 14, wherein said yeast selection marker pair further comprises a yeast selection marker selected from the group consisting of a LEU2 gene, a HIS3 gene, an ADE2 gene, a LYS2 gene and a kanMS6 gene.

16. The vector of claim 12, wherein each of said at least one pair of yeast selection markers further comprise at least one bacterial selection marker.

17. The vector of claim 17, wherein said bacterial selection marker is selected from at least one member of the group consisting of an ampicillin resistance gene, a tetracycline resistance gene, a chloramphenicol resistance gene and a gentamycin resistance gene.

18. A method for making a recombinant plastid genome comprising, constructing a vector of claim 1, introducing said vector into yeast, inserting, deleting or both inserting and deleting at least one nucleotide sequence into or from said vector by homologous recombination to produce a modified vector containing said recombinant plastid genome, isolating said modified vector from said yeast, introducing said isolated modified vector into bacteria, and amplifying said modified vector in said bacteria.

19. The method of claim 18, wherein said plastid genome is a chloroplast genome.

20. The method of claim 19, further comprising introducing said modified genome into a chloroplast of a vascular plant.

21. The method of claim 20, wherein said chloroplast is incapable of photosynthesis or made incapable of photosynthesis prior to introduction of said modified genome.

22. The method of claim 21, wherein said chloroplast is capable of photosynthesis after introduction of said modified genome.

23. The method of claim 18, wherein said at least one nucleotide sequence codes at least one exogenous protein.

24. A host cell comprising a vector of claim 1.

25. The host cell of claim 1, wherein said host cell is a bacterial cell or a yeast cell.

26. The host cell of claim 1, wherein said host cell is a cell of a vascular plant.

27. A modified plastid genome produced by the method of claim 18.

28. The plant comprising the modified plastid genome of claim 27.


SEQUENCE LISTING (67 sequences)

SEQ ID NO: 1 - DNA, 22908 bp, Artificial Sequence
  Synthetic polynucleotide
tgaatttatcagatctaactgaggagtaagaaacccccatgtcaaagaaaaacagaccaacaattgggcgaacccttaatccttcaatattaagcggatttgatagttcttcagcctctggcgatcgagtcgagcaggtattcaagttatcaactggtcgccaggccacatttattgaagaggtaatacctccgaaccaggtagaaagcgatacctttgttgatcagcataacaacgggcgtgaccaggcatctcttacgccaaaatcattaaaaagtatccgaagcactattaagcatcagcaattttaccctgcaataggtgttagacgggctacagggaaaattgaaattttggatggttcccggcgtcgagcttctgccatcttagagaacgtagggttgcgggttttagtcacggaccaggagatcagcgttcaggaagcgcaaaatttagcgaaagacgttcagacagcattgcagcacagcattcgagaaataggtctgcgtttgatgcgaatgaaaaatgatgggatgagtcagaaggatattgcagccaaagaagggctgtctcaggcgaaggtcacgcgtgctctccaggcagcgagtgctccggaagaattagtcgcccttttccctgtgcagtcggaattaaccttttcggactacaaaacgctttgtgctgttggcgacgaaatggggaacaagaatttagagtttgatcagcttattcaaaacatatccccggaaataaacgacatcttatccattgaagaaatggccgaagatgaagttaaaaataaaatcctgcgcttgataacaaaggaagcctcactactcacggataaaggttctaaagataagtccgtagttactgaattatggaaatttgaggacaaggatcgctttgcaaggaagcgcgtgaaaggccgtgcattttcttatgagtttaatcgactctcaaaagagttacaggaagaactcgacaggatgattgggcatatccttagaaagagcctcgataaaaagccgaagccttaaactttcgccattcaaatttcactattaactgactgtttttaaagtaaattactctaaaatttcaaggtgaaatcgccacgatttcaccttggattttaccttcctcccctcctcccgaaaaaaataaaaaaattgcttgtcacgagaaagtcaacaagtgactttcaataaaatctcttccgaaaagggattcacacaagtgccttgtgtttaaggaagagtaaattgagtaacttacgcgaataccagaatcgtattgcagatatcgcaaaacgctctaaagctgtgcttggctgggcaagcactgcgcagttcggtactgataaccaattcattaaagatgatgccgcgcgtgccgcatctatccttgaagctgcacgtaaagacccggtttttgcgggtatctctgataatgccaccgctcaaatcgctacagcgtgggcaagtgcactggctgactacgccgcagcacataaatctatgccgcgtccggaaattctggcctcctgccaccagacgctggaaaactgcctgatagagtccacccgcaatagcatggatgccactaataaagcgatgctggaatctgtcgcagcagagatgatgagcgtttctgacggtgttatgcgtctgcctttattcctcgcgatgatcctgcctgttcagttgggggcagctaccgctgatgcgtgtaccttcattccggttacgcgtgaccagtccgacatctatgaagtctttaacgtggcaggttcatcttttggttcttatgctgctggtgatgttctggacatgcaatccgtcggtgtgtacagccagttacgtcgccgctatgtgctggtggcaagctccgatggcaccagcaaaaccgcaaccttcaagatggaagacttcgaaggccagaatgtaccaatccgaaaaggtcgcactaacatctacgttaaccgtattaagtctgttgttgataacggttccggcagcctacttcactcgtttactaatgctgctggtgagcaaatcactgttacctgctctctgaactacaacattggtcagattgccctgtcgttctccaaagcgccggataaaggcactgagatcgcaattgagacggaaatcaatattgaagccgctcctgagctgatcccgctgatcaaccacgaaatgaagaaatacaccctgttcccaagtcagttcgttatcgcggctgagcacacggtacaggcggcgtatgaagcacagcgtgaatttggtctggacctgggttccctacagttccgcaccctgaaggaatacctgtctcatgaacaggatatgctgcgtcttcgcatcatgatctggcgcactcttgcgaccgacacctttgacatcgctctgccggttaaccagtcctttgatgtatgggcaaccatcattcgtggcaaattccagactgtatatcgcgacattattgagcgcgttaaatcttctggtgcgatggggatgtttgctggtgctgatgcagcatctttcttcaaacagttgccgaaggatttcttccagccagccgaagactatatccagactccgtatgttcactacatcggtaccccatttaggaccacccacagcacctaacaaaacggcatcagccttcttggaggcttccagcgcctcatctggaagtggaacacctgtagcatcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtagatatactagaagttctcctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctacaactccacttattgttaggtagaattgtccgttagttgtttattaattgcaataatggggcgtccagttttggcaacagtgtcctcttaccaggacacctatgagtttgcctcatggcaaactagaggtgttgaaagtatgcatggttataattagagcaattcattaccctctgaatcctgccggtataccccattgttcgttatctttatttttggctaaaaccgcattaagagcttcgtttaccgtcatgcaatgcggtaggttatcgaagtttgatatcccgccaatatcaggcgaacgcttgttcttcaggtaagcatatttccgcgcagccgcctctactttctgcttgaactcatgtttttgagtgcgttttttggataaccgcagattgtcagcctttgcttttgccttagcgatccatgaagtcaattttttgaggctggttgttccggcaccgccggaaactgatctttttgtttttttaacttgtgacttcttattctttattgccacgtcatcctgacagggggagggggtatcattttgacatgggggtgtggataaaaaattaaataaagccaatgtcttagcgagaacagctttaaccttggttgccgctgaagagatctttaatttgctttctatcagcgcatttttggcttgttgtgcgaaggccaaaaaggatggtgtaaaccggtacaggttagcgcgacgttcacggtgatcgccgataacaatctctacagacagaattcctttgtttacagcttcacggaatgcacgaacgacggttgattggctataaccagtttctgccgcgatcaggcggtgaggcttgtgaatgaagtattcactggttgttgccgcgagatttgcacattgcgacaggatatgcccggcgctacgggatagaccggagtgtgttacaaagcaggccaattcatagccagaaaaagtaaaatcgctcatcgttatacagctcaggaaagtgactttagccagcattacaatgctggtggttcttactacgtctgttagcgcgttgccgcgacaggtaccagcacaccagcatcaagcaatcgcttcatcagccactgctgacctttgccggttatacgagtcgtgaaagaaatcctgcttccattgcttgtatcgatcacggtttctttaagggtgaaatacccacgagatatgtattcttgtttggggacgttcctgcgttcaccggttgcgatcagaattccgttatcacgcaaccaggtgaagagatagttttggcccaggccgagcactttggcatagttgccgattagaaccccgctggcggtagcaacgcgttcagcgaattcgactttaggtgcatccataagcattttttgctccagccgttgcttttgctctgccaggtcggcagccaaacggagagcttcagggagactctgcggaatagcaggttgtaatcttccggttcgatagtcgataaatgtctggtttaccttcagccgaaacgcgggagaaatccagcctgcgtactccacagcgagcaattcatgggcaaaagtgccgccgccacggccttcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggtagtcgatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaggctctcaagggcatcggtcgagcttgacattgtaggacgtttaaacattaccctgttatccctaggatcctacgtaatcgatgaattcgatcccatttttataactggatctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcgtatggaattgcccaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaatttatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagaccttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattgattccaagcattatctaaaatactctgcaggcacgctagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttatttttatggtgtttaggttaaatacctaaacatcattttacatttttaaaattaagttctaaagttatcttttgtttaaatttgcctgtctttataaattacgatgtgccagaaaaataaaatcttagctttttattatagaatttatctttatgtattatattttataagttataataaaagaaatagtaacatactaaagcggatgtagcgcgtttatcttaacggaaggaattcggcgcctacgtatacatactccgaaggaggacaaatttatttattgtggtacaataaataagtggtacaataaataaattgtatgtaaaccccttccccttcgggacgtccccttacgggaatataaatattagtggcagttgcctgccaacaaatttatttattgtattaacataggcagtggcggtaccactgccactggcgtcctaatataaatattgggcaactaaagtttatcgcagtattaacataggcagtggcggtaccactgccactggcgtcctccttcggagtatgtaaacctgctaccgcagcaaataaattttattctattttaatactacaatatttagattcccgttaggggataggccaggcaattgtcactggcgtcatagtatatcaatattgtaacagattgacaccctttaagtaaacattttttttaggattcatatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcaatgacaattgcgatcggtacatatcaagagaaacgcacatggttcgatgacgctgatgactggcttcgtcaagaccgtttcgtattcgtaggttggtcaggtttattactattcccttgtgcttactttgcaactccggtccggcggccgcctcgagacgacttgtccgcttcatcagacacggctttcctaaccatcaatggtggattttcaggaaagacgtttaaagaagtggcataaagtttatttgttgaagaattggttttgtttccattcaaagaattgttagggataaaactttgcatttttttataatttgttataagtttttcaaacttatatgtttttaaaaatgcatttaattgcttattaatgcgttcattttgtaatgtttcaataggtcttgcttgcgctaatcgcagtattcccgatactttgtctgcttgtttttcgggtattgagaataagtaagtataatgatttaaaaaagtcatgttttgattaaatcttttttatatggttaaaaacattatggtatatctaaataaatttattttttactaaatctccaatttgcaatttagagatataattaaaactataaagttatttaagttaatttgtaatcaaatccaacacaaaaatgtttttatatagttaacatgttaaatttaacatatgttaaacaactaaaattctgtaacagagaacaataaaataaatgctagattttgtgtaatgccgaagtatatttatatacttccctttcaaaaaaataaatactcttgccactaaaattcatttgcctaggacgtccccttccccttacgggatgtttatatactaggacgtccccttccccttacgggatatttatatactccgaaggacgtccccttcgggcaaataaattttagtggcagttgcctgccaactgcctaggcaagtaaacttagggattttaatgcaataaataaatttgtccccttacgggacgtcagtggcagttgcctgccaactgcctaatataaatattagtggatatttatatactccgaaggaggcagttacctgccaactgccgaggcaaataaattttagtggcagtggtaccgccactgcctgctccctccttccccttcgggcaagtaaacttagcatgttgtcgacattaccctgttatccctaggccggcctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaatgaaatgagatgatacttgcttatctcatagttaactggcataaattttagtataggttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgatttaggcaaaagttactatttctggctcgtgtaatatatgtatgctaatgtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatattaggacttccacaccaactagtaatatgacaataaattcaagatattcttcatgagaatggcccagctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcaattctttagtataaatttcactctgaaccatcttggaaggaccggataattatttgaaatctctttttcaattgtatatgtgttatgtagtatactctttcttcaacaattaaatactctcggtagccaagttggtttaaggcgcaagactttaatttatcactacggaattggcctattaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactacgtcgttaaggccgtttctgacagagtaaaattcttgagggaactttcaccattatgggaaatggttcaagaaggtattgacttaaactccatcaaatggtcaggtcattgagtgttttttatttgttgtattttttttttttagagaaaatcctccaatatataaattaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccgcgcttgcggaagcatcagcaaataaggccagcacagccagcgcagttgccgctttggttcctgattctgttcttgatgaattaaacaaagcggcacagtaacaaaggacttcattgataatttttcttcaggaggaagacatgtcattcttttctacgttaaaaacagctttgtctttgaaggagaaacttgctgctactggtgttcttgttctgatttgcgcacttgttggtgctgggtttgcatgggaacgtcatcagctaaagcaagccatagagaaaattggcagtcttgatcaggctgttaaggaacgtgataagtcaataatggatcttaaccagaccattgagacgatgaacaaagcagagcaacattttcacagccaggaagtgaaaaatgaatcagaacaagccaagtatgctgacaggcaaatggaacgaaaagctgaagttcagaaacaactggttgcggcgggtaatgttcgccagcgtattcctgctgacactcagcggttgctccgggagtcgatcagcgaatttaacgccgacgccgacaaaggttaaccaccctgcccccaaaagtgcatttatgtgtaggatgccagagtttagcagtgaatattttgatgatctgccagcgtatatcctcgatacagaaacgatgctgatggggattaacaggaagaatcgcaacgttaatgattacaaccgagctattagcggtaactaaaagggatttttatgtctgataaagtaacagtaaagcaaactatcaacaaagcgacttcaatctacaaaattgagcaaatcactgttggcaagccaggatctgaacaataccgtcgtgctttcgagcttgccgatcagcttggtttaaaacacccggattgcattgagcatgtatttccgacctatgctgatgagcaatgtactcatgttcttaccgaagaggattttttcagcactgaagaacgagaaggcgttgatcgctgcattggtgtgatttgttcttcggtaagtgatgagttattccctaatgtgcctgaatatggtggtattggataccaattcctgtacgagggcgatgagcttaaatgctatgaacatggtcttctcatcgaaagcgtagaataatacgactcccttccaaccggctacgttggccggtttttcacttatccacattatccactggatagatccaataatcaggtccatacagatcccaattagatccatatagatccctgatcgttgcaggccgcgccacgtctggcttagaagtgtatcgcgatgtgtgctggagggaaaacgatgtgtgctggagggataaaaatgtgtgctgacgggttgctaatgtgtgctggcgggatataggatgtgtgttgacgggaaagcttgggtagttatcaccacttataaaaactatccacacaattcggaaaaagtaatatgaatcaatcatttatctccgatattctttacgcagacattgaaagtaaggcaaaagaactaacagttaattcaaacaacactgtgcagcctgtagcgttgatgcgcttgggggtattcgtgccgaagccatcaaagagcaaaggagaaagtaaagagattgatgccaccaaagcgttttcccagctggagatagctaaagccgagggttacgatgatattaaaatcaccggtcctcgactcgatatggatactgatttcaaaacgtggatcggtgtcatctacgcgttcagcaaatacggcttgtcctcaaacaccatccagttatcgtttcaggaattcgctaaagcctgtggtttcccctcaaaacgtctggatgcgaaactgcgtttaaccattcatgaatcacttggacgcttgcgtaacaagggtatcgcttttaagcgcggaaaagatgctaaaggcggctatcagactggtctgctgaaggtcgggcgttttgatgctgaccttgatctgatagagctggaggctgattcgaagttgtgggagctgttccagcttgattatcgcgttctgttgcaacaccacgccttgcgtgcccttccgaagaaagaagctgcacaagccatttacactttcatcgaaagccttccgcagaacccgttgccgctatcgttcgcgcgaatccgtgagcgcctggctttgcagtcagctgttggcgagcaaaaccgtatcattaagaaagcgatagaacagcttaaaacaatcggctatctcgactgttctattgagaagaaaggccgggaaagttttgtaatcgtccattctcgcaatccaaagctgaaactccccgaataagtgtgtgctggagggaaaccgcattaaaaagatgtgtgctgccgggaaggcttgtccaatttcctgtttttgatgtgcgctggagggggacgcccctcagtttgcccagactttccctccagcacacatctgtccatccgcttttccctccagtgcacatgtaattctctgcctttccctccagcacacatatttgataccagcgatccctccacagcacataattcaatgcgacttccctctatcgcacatcttagacttttattctccctccagcacacatcgaagctgccgggcaagccgttctcaccagttgatagagagtgaagcttggctgcccattgaagcaggaaatcaccaaaatgattcaggctacaacctgaacgtagaagaaatccgcgtcctttatgcgtggaggatgccaaagcatgttgtgacacacttggcaaaggagtaagcatgcagagaatgctatgtacaagcatctacgcatacattattattttatgcagcatttttaattaaattcaaaaatacagcataaaggatgactttcgatgagtgattccagccagcttcacaaggttgctcaaagagcaaacagaatgctcaatgttctgactgaacaagtacagttgcaaaaggatgagctacacgcgaacgagttttaccaggtctatgcgaaagcggcactggcaaaattgcctctactgactcgagcgaacgttgactatgccgtaagtgaaatggaagaaaagggttatgttttcgataaacgccctgctggctcttcaatgaaatatgcgatgtcaattcagaacatcattgacatatatgaacatcgcggagtgccaaaataccgggatcgctacagcgaagcgtatgtgattttcatctccaatcttaaaggcggtgtgtcaaaaactgtatcgacggtttctctggcgcatgcaatgcgtgctcaccctcatcttcttatggaggatttaaggattctggttattgaccttgatccgcaatcttcagcaacgatgtttttaagccataaacactctattggtatcgtaaacgcaacatctgcacaggctatgttgcagaatgtaagccgtgaagagctgttagaggagtttattgttccttctgttgtacctggggttgacgttatgcctgcgtcgattgacgatgcctttattgcatccgattggagagagctgtgcaatgagcatctaccgggtcagaacatccatgctgtcctgaaagaaaatgtgattgataagctgaagagcgattatgactttatcctcgttgatagtggtcctcaccttgacgccttcctgaaaaatgctttggcctcggccaatatactgtttacacctctgccgccagcaactgtcgatttccactcatcgcttaaatacgttgcccgccttcctgagttggtgaaactcatttcggatgaaggctgcgagtgccagcttgcgactaacattggttttatgtccaagttgagtaacaaggcagaccataagtattgccatagcctggctaaagaagtgttcggtggggatatgcttgatgtcttcctccctcgccttgacggttttgaacgctgcggcgagtcttttgacactgttatttcagctaacccggcaacgtatgttggtagtgctgatgcattgaagaacgcgcgaattgccgcggaagattttgctaaagcagtttttgaccgtat

SEQ ID NO: 2 - DNA, 70 bp, Artificial Sequence
  Primer
tgacagcttatcatcgaatttctgccattcatccgcttattatcactaagaaaccattattatcatgaca

SEQ ID NO: 3 - DNA, 70 bp, Artificial Sequence
  Primer
ggcagttattggtgcccttaaacgcctggttgctacgcctgaataaaattccgtagtgataaattaaagt

SEQ ID NO: 4 - DNA, 7507 bp, Artificial Sequence
  Synthetic polynucleotide
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 5 - DNA, 44 bp, Artificial Sequence
  Primer
ttggttggtacgtactcgagtgcaagcttgagtattctatagtg

SEQ ID NO: 6 - DNA, 44 bp, Artificial Sequence
  Primer
ttggttggtacgtactcgagtccattgctgacataatccgctcc

SEQ ID NO: 7 - DNA, 2474 bp, Artificial Sequence
  Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagggcggccgccagctggaattctacgtactgcagagtactgcggccgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 7453 bp, Artificial Sequence
  Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagggcggccgtactcgagtgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcactaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaatgaaatgagatgatacttgcttatctcatagttaactggcataaattttagtataggttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgatttaggcaaaagttactatttctggctcgtgtaatatatgtatgctaatgtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatattaggacttccacaccaactagtaatatgacaataaattcaagatattcttcatgagaatggcccagctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcaattctttagtataaatttcactctgaaccatcttggaaggaccggataattatttgaaatctctttttcaattgtatatgtgttatgtagtatactctttcttcaacaattaaatactctcggtagccaagttggtttaaggcgcaagactttaatttatcactacggaattttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggactcgagtacggccgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 1353 bp, Saccharomyces cerevisiae
tcgcgcgtttcggtgatgacggtgaaaacctctgacacatgcagctcccggagacggtcacagcttgtctgtaagcggatgccgggagcagacaagcccgtcagggagcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcagga

SEQ ID NO: 10 - DNA, 2474 bp, Saccharomyces cerevisiae
tcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 11 - DNA, 1177 bp, Saccharomyces cerevisiae
aattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcg

SEQ ID NO: 12 - DNA, 3008 bp, Saccharomyces cerevisiae
tgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaacccccccccccccccccgatgcgccgcgtgcggctgctggagatggcggacgcgatggatatgttctgccaagggttgggtcgacgctaccttaagagagacgcgtgcggccgcaagcttgcatgcctgcaggtcgatcgactctagaaatcgatagatctgaattaattcttgaataatacataacttttcttaaaagaatcaaagacagataaaatttaagagatattaaatattagtgagaagccgagaattttgtaacaccaacataacactgacatctttaacaacttttaattatgatacatttcttacgtcatgattgattattacagctatgctgacaaatgactcttgttgcatggctacgaaccgggtaatactaagtgattgactcttgctgaccttttattaagaactaaatggacaatattatggagcatttcatgtataaattggtgcgtaaaatcgttggatctctcttctaagtacatcctactataacaatcaagaaaaacaagaaaatcggacaaaacaatcaagtatggattctagaacagttggtatattaggagggggacaattgggacgtatgattgttgaggcagcaaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcagattgatacaagacaaatatattcaaaaagagcatttaatcaaaaatggtatagcagttacccaaagtgttcctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatgggcaccatttactaaagaattagcagtcatgattgtgaggtctgttaacggtttagtgttttcttacccaattgtagagactatccacaaggacaatatttgtgacttatgttatgcgcctgctagagttccggactccgttcaacttaaggcgaagttgttggcagaaaatgcaatcaaatcttttcccggttgtggtatatttggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacaggtagaactgatattccaatcaaaatctctgtcgctcaaaagttggacttggaagcaatggtcaaaccattggttggaatcatcatgggatcagactctgacttgccggtaatgtctgccgcatgtgcggttttaaaagattttggcgttccatttgaagtgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaattgtgcaaatgcctagaggtgttccagtagctaccgtcgctattaataatagtacgaacgctgcgctgttggctgtcagactgcttggcgcttatgattcaagttatacaacgaaaatggaacagtttttattaaagcaagaagaagaagttcttgtcaaagcacaaaagttagaaactgtcggttacgaagcttatctagaaaacaagtaatatataagtttattgatatacttgtacagcaaataattataaaatgatatacctattttttaggctttgttatgattacatcaaatgtggacttcatacatagaaatcaacgcttacaggtgtccttttttaagaatttcatacataagatctctcgaggatccccgggtaccgagctcgaattcgcggccgcccgcgggttaaccctagggcatgcactagtggcctaattggccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaa

SEQ ID NO: 13 - DNA, 4879 bp, Saccharomyces cerevisiae
agcagttgctttctcctatgggaagagctttctaagtctgaagaagtaaacagttctttgctatttcacacttcctggttgatggtcacttgctgcctgaaatatatatatatgtatgacatatgtacttgttttcttttttgtgcctttgttacgtctatattcattgaaactgattattcgattttcttcttgctgaccgcttctagaggcatcgcacagttttagcgaggaaaactcttcaatagttttgccagcggaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgta

SEQ ID NO: 14 - DNA, 1475 bp, Artificial Sequence
  Synthetic polynucleotide
ttaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaata

SEQ ID NO: 15 - DNA, 36 bp, Artificial Sequence
  Primer
ttggttcccgggtcgcgcgtttcggtgatgacggtg

SEQ ID NO: 16 - DNA, 42 bp, Artificial Sequence
  Primer
ttggttgtcgacccgcggtgatgcggtattttctccttacgc

SEQ ID NO: 17 - DNA, 36 bp, Artificial Sequence
  Primer
ttggttcccgggtcctgatgcggtattttctcctta

SEQ ID NO: 18 - DNA, 36 bp, Artificial Sequence; identical to SEQ ID NO: 15
  Primer

SEQ ID NO: 19 - DNA, 36 bp, Artificial Sequence
  Primer
ttggttcccgggaagcttgcatgcctgcaggtcgat

SEQ ID NO: 20 - DNA, 36 bp, Artificial Sequence
  Primer
ttggttcccgggagcagttgctttctcctatgggaa

SEQ ID NO: 21 - DNA, 36 bp, Artificial Sequence
  Primer
ttggttcccgggttaggtctagagatctgtttagct

SEQ ID NO: 22 - DNA, 50 bp, Artificial Sequence
  Primer
ttggttgtcgacggccggccactagttcgcgcgtttcggtgatgacggtg

SEQ ID NO: 23 - DNA, 50 bp, Artificial Sequence
  Primer
ttggttgtcgacggccggccactagttgatgcggtattttctccttacgc

SEQ ID NO: 24 - DNA, 50 bp, Artificial Sequence
  Primer
ttggttgtcgacggccggccactagtgatcctcgagagatcttatgtatg

SEQ ID NO: 25 - DNA, 50 bp, Artificial Sequence
  Primer
ttggttgtcgacggccggccactagttacaataaaccaagatgaagctgc

SEQ ID NO: 26 - DNA, 50 bp, Artificial Sequence
  Primer
ttggttgtcgacggccggccactagttattaagggttctcgagagctcgt

SEQ ID NO: 27 - DNA, 4024 bp, Artificial Sequence
  Synthetic polynucleotide
tatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgcggccgcctcgagacgccgtaaacagataggaatggacgttgcctagaaagaatcaattttagtggacgtccccttacgggacaataaattaatttgttgcctcgcctatcggctaacaagtttggcagtggcaggccactgccactgacgcccttccccttccccttcgggacaataaataaatttgttgcctgccaacaaatttatttattgtattaacattgtattaacatagaatgtttacatactccgaaggaggacaaatttatttattgtggtaccgccactgcctgcttcctccttccccttccccttcggggatataaatatagggcaagtaaacttagcataaactttagttacccaatatttatatactccgaaggacgtcagtggcagttgcctgccaactgcctatgcaactaaagtttatcgcagtatataaatatagaataaaatttatttgctgcgctagcaggtttacatactcccaagtttacttgcccgaaggggaaggaggacgtccccttacgggacaataaataaatttgttgcctgccaacaaatttatttattgtattaacatcctaatataaatattagtggacgtccccttcgggcaaatgaattttagtggatatttatatactccgaaggaggcagttgcctgccaactgcctaggcaagtaaacttaggagtattaaaataggacgccagtggcagtggtaccgccactgcctatgttaatactgcgataaactttagttgcccgaaggggtttacatacgtcgacattaccctgttatccctaggatcctacgtacagtggcagtggtaccgccactgcctagtatgtaaacattctatatttatatactcctaagtttacttgcccgaaggggaaggaggcaactgccactaaaattcatttgcccgaaggggacgtccacttactgaattactatttattttctctagagcaagaaaatagctgttgagcggtttagaccttttctggtctgccatatacatgtaacaagctaacctaaagaataacattcaactataaaactaaatgattttattttatgattagcatgttttttcctaaaatatatttatttgacataaatatatttatgtgatataatatatttaaatgtatttaaaatttttcaacaatttttaaattatatttccggacagattattttaggatcgtcaaaagaagttacatttatttagaactatgatacaaaataccatcatgctaccaatggtagtaactggtttgtttttagttggttattattattattataaggcagtgatgcaattacaatacactccaaccagttatgtaattgattcaactgcattagagttttatagtagtcaatctacgtatactttaaagaaaattttactagctcatgcctgtaacgttaataggttatgcaccttggactttcaagccagtgccacggcttatttaaatcaggtggaagcttccgggtttttacaaggctttggcttgttaggaaacttattgtgtttattagatggggcggatgactccggtccggcggccgccctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaac

SEQ ID NO: 28 - DNA, 1382 bp, Artificial Sequence
  Synthetic polynucleotide
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacat

SEQ ID NO: 29 - DNA, 679 bp, Artificial Sequence
  Synthetic polynucleotide
cccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtga

SEQ ID NO: 30 - DNA, 39 bp, Artificial Sequence
  Primer
ttggttcccggggatatcaatacattcaaatatgtatcc

SEQ ID NO: 31 - DNA, 41 bp, Artificial Sequence
  Primer
ttggttcccggggatatcatccttttaaattaaaaatgaag

SEQ ID NO: 32 - DNA, 42 bp, Artificial Sequence
  Primer
ttggttcccggggatatcttctcatgtttgacagcttatcat

SEQ ID NO: 33 - DNA, 42 bp, Artificial Sequence
  Primer
aaccaacccggggatatcatgttctgccaagggttggtttgc

SEQ ID NO: 34 - DNA, 42 bp, Artificial Sequence
  Primer
ttggttcccggggatatccccaatccaggtcctgaccgttct

SEQ ID NO: 35 - DNA, 42 bp, Artificial Sequence
  Primer
aaccaacccggggatatctcacttattcaggcgtagcaacca

SEQ ID NO: 36 - DNA, 42 bp, Artificial Sequence
  Primer
ttggttcccggggatatcttgccgggtgacgcacaccgtgga

SEQ ID NO: 37 - DNA, 42 bp, Artificial Sequence
  Primer
aaccaacccggggatatctgccgatggccgcggcgttgtgac

SEQ ID NO: 38 - DNA, 60 bp, Artificial Sequence
  Primer
aaaaatgaatttgaattctaattgaatatgtttgttgttttggggtaataactgatataa

SEQ ID NO: 39 - DNA, 60 bp, Artificial Sequence
  Primer
accccttgaatcactactacagtacttgattcaaggggttctggaatcatagtttcatga

SEQ ID NO: 40 - DNA, 20 bp, Artificial Sequence
  Primer
acaatccactgccttgatcc

SEQ ID NO: 41 - DNA, 20 bp, Artificial Sequence
  Primer
gctatgcatggttccttggt

SEQ ID NO: 42 - DNA, 21 bp, Artificial Sequence
  Primer
gcttccattgagtctctgcac

SEQ ID NO: 43 - DNA, 22 bp, Artificial Sequence
  Primer
gccattcgattcgttttagttc

SEQ ID NO: 44 - DNA, 20 bp, Artificial Sequence
  Primer
aacccattcttaccccaagc

SEQ ID NO: 45 - DNA, 20 bp, Artificial Sequence
  Primer
acggctcagcagtcaattct

SEQ ID NO: 46 - DNA, 23 bp, Artificial Sequence
  Primer
aagatcggactacaatcaccgta

SEQ ID NO: 47 - DNA, 22 bp, Artificial Sequence
  Primer
gggatcaaagtacccattttgt

SEQ ID NO: 48 - DNA, 20 bp, Artificial Sequence
  Primer
caaaagacggggagactcaa

SEQ ID NO: 49 - DNA, 21 bp, Artificial Sequence
  Primer
tgggcatgtacttggaaactc

SEQ ID NO: 50 - DNA, 20 bp, Artificial Sequence
  Primer
ctaacccgaagtgctccttg

SEQ ID NO: 51 - DNA, 20 bp, Artificial Sequence
  Primer
tgtcgagaagtgcaaaatcg

SEQ ID NO: 52 - DNA, 20 bp, Artificial Sequence
  Primer
tttccggaatatgagtgtgc

SEQ ID NO: 53 - DNA, 20 bp, Artificial Sequence
  Primer
tatcaagtcggctccttcgt

SEQ ID NO: 54 - DNA, 25 bp, Artificial Sequence
  Primer
cgacgagatctattatcatttttcg

SEQ ID NO: 55 - DNA, 20 bp, Artificial Sequence
  Primer
aaaactgctcggcaacagtc

SEQ ID NO: 56 - DNA, 24 bp, Artificial Sequence
  Primer
tgcaatagaaaaagaatccagaaa

SEQ ID NO: 57 - DNA, 21 bp, Artificial Sequence
  Primer
agtttgctgtatgcggactaa

SEQ ID NO: 58 - DNA, 20 bp, Artificial Sequence
  Primer
gggaaaacgacaaagattgc

SEQ ID NO: 59 - DNA, 20 bp, Artificial Sequence
  Primer
tagaagaagggggtttgtgc

SEQ ID NO: 60 - DNA, 20 bp, Artificial Sequence
  Primer
cttctgccccaagaacagag

SEQ ID NO: 61 - DNA, 20 bp, Artificial Sequence
  Primer
tcgaattcccacatgatgaa

SEQ ID NO: 62 - DNA, 21 bp, Artificial Sequence
  Primer
ccggaagatatatccacatcg

SEQ ID NO: 63 - DNA, 20 bp, Artificial Sequence
  Primer
cccgcctcctacatatttga

SEQ ID NO: 64 - DNA, 20 bp, Artificial Sequence
  Primer
ggggccacgagaattattta

SEQ ID NO: 65 - DNA, 20 bp, Artificial Sequence
  Primer
ctgttgacattgcgaagagc

SEQ ID NO: 66 - DNA, 21 bp, Artificial Sequence
  Primer
tgtcgccgaagaagttaagaa

SEQ ID NO: 67 - DNA, 20 bp, Artificial Sequence
  Primer
tgggatccaaaggaaacttg